(19) United States
(12) Patent Application Publication
(10) Pub. No.: US 2024/0277927 A1
(43) Pub. Date: Aug. 22, 2024

(54) Article, System, and Method for Detecting Extravasation

(71) Applicant: 3M INNOVATIVE PROPERTIES COMPANY, St. Paul, MN (US)

(72) Inventors: Dawn V. Muyres, Stillwater, MN (US); Stephen M. Kennedy, St. Paul, MN (US); Minghua Dai, Plymouth, MN (US)

(21) Appl. No.: 18/571,379

(22) PCT Filed: Jun. 24, 2022
(86) PCT No.: PCT/IB2022/055901

§ 371 Date: Dec. 18, 2023

Related U.S. Application Data
(60) Provisional application No. 63/214,875, filed on Jun. 25, 2021.

(51) Int. Cl. A61M 5/168; A61B 5/00; A61B 5/053; A61B 5/262

(52) U.S. Cl. CPC A61M 5/16836; A61B 5/053; A61B 5/262; A61B 5/746

(57) ABSTRACT

The present disclosure provides an article for detecting extravasation into a tissue. The article includes a body having a first major surface, an opposing second major surface, a first side, and an opposing second side. The article further includes a first electrode disposed on the first major surface of the body. The first electrode includes at least one skin-penetrating microfeature. The article further includes a second electrode disposed on the first major surface of the body. The second electrode includes at least one skin-penetrating microfeature. The first electrode is electrically connected to the second electrode.

### TECHNICAL FIELD

The present disclosure relates generally to an article, a system, and a method for detecting extravasation, and in particular to an article, a system, and a method for detecting extravasation based on impedance monitoring.

### BACKGROUND

Extravasation or infiltration is an accidental infusion or leakage of a fluid, such as a contrast medium or a therapeutic agent, into a tissue surrounding a vein rather than into the vein itself. Extravasation may be caused by, for example, rupture or dissection of fragile vasculature, valve disease, inappropriate needle placement, and/or patient movement.

### SUMMARY

In one aspect, the present disclosure provides an article for detecting extravasation into a tissue. The article includes a body. The body includes a first major surface, an opposing second major surface, a first side, and an opposing second side. The article further includes a first electrode disposed on the first major surface of the body. The first electrode includes at least one skin-penetrating microfeature. The article further includes a second electrode disposed on the first major surface of the body. The second electrode includes at least one skin-penetrating microfeature. The first electrode is electrically connected to the second electrode.

In another aspect, the present disclosure provides a system for detecting extravasation into a tissue. The system includes an article and a controller. The article includes a body. The body includes a first major surface, an opposing second major surface, a first side, and an opposing second side. The article further includes a first electrode disposed on the first major surface of the body. The first electrode includes at least one skin-penetrating microfeature. The article further includes a second electrode disposed on the first major surface of the body. The second electrode includes at least one skin-penetrating microfeature. The first electrode is electrically connected to the second electrode. The controller is electrically connected to the first electrode and the second electrode. The controller is configured to provide an input signal across the first electrode and the second electrode. The controller is further configured to determine an output signal across the first electrode and the second electrode in response to the input signal. The controller is further configured to determine at least one electrical parameter based on the output signal and the input signal. The controller is further configured to detect extravasation of a fluid into the tissue based on a change in the at least one electrical parameter.

In yet another aspect, the present disclosure provides a method for detecting extravasation. The method includes providing an article including a body, a first electrode including at least one skin-penetrating microfeature disposed on a first major surface of the body, and a second electrode including at least one skin-penetrating microfeature disposed on the first major surface of the body. The second electrode is spaced apart from the first electrode. The method further includes placing the first major surface of the body on an injection site. The injection site is disposed between the first electrode and the second electrode. The method further includes providing an input signal across the first electrode and the second electrode. The method further includes determining an output signal across the first electrode and the second electrode in response to the input signal. The method further includes determining at least one electrical parameter based on the output signal and the input signal. The method further includes determining extravasation into a tissue based on a change of the at least one electrical parameter.

### BRIEF DESCRIPTION OF THE DRAWINGS

Exemplary embodiments disclosed herein may be more completely understood in consideration of the following detailed description in connection with the following figures. The figures are not necessarily drawn to scale. Like numerals used in the figures refer to like components. However, it will be understood that the use of a numeral to refer to a component in a given figure is not intended to limit the component in another figure labeled with the same number.

FIG. 1A illustrates a schematic bottom view of a conventional sensor patch;

FIG. 1B illustrates a graph depicting a percentage change of an impedance magnitude with respect to an extravasated volume of a fluid determined using the conventional sensor patch of FIG. 1A;

FIG. 2 illustrates a schematic sectional side view of an article for detecting extravasation into a tissue according to an embodiment of the present disclosure;

FIG. 3A illustrates a schematic top view of an article for detecting extravasation into a tissue according to another embodiment of the present disclosure;

FIG. 3B illustrates a schematic sectional side view of the article of FIG. 3A;

FIG. 4 illustrates a schematic top view of an article for detecting extravasation into a tissue according to another embodiment of the present disclosure;

FIG. 5A illustrates a schematic bottom perspective view of a system for detecting extravasation into a tissue according to an embodiment of the present disclosure;

FIG. 5B illustrates a schematic block diagram of a system for detecting extravasation into a tissue according to another embodiment of the present disclosure;

FIG. 5C illustrates a graph depicting a variation of an extravasated volume of fluid with respect to time according to an embodiment of the present disclosure;

FIG. 6A illustrates a graph depicting a percentage change of an electrical parameter with respect to an extravasated volume of a fluid according to an embodiment of the present disclosure;

FIG. 6B illustrates a graph depicting a percentage change of another electrical parameter with respect to an extravasated volume of a fluid according to an embodiment of the present disclosure;

FIG. 6C illustrates a graph depicting a percentage change of another electrical parameter with respect to an extravasated volume of a fluid according to an embodiment of the present disclosure;

FIG. 7 illustrates a flowchart depicting various steps of a method for detecting extravasation according to an embodiment of the present disclosure;

FIG. 8A illustrates a graph depicting a variation of impedance magnitude with respect to frequency for a pair of wet electrodes;

FIG. 8B illustrates a graph depicting a variation of percentage change of impedance magnitude with respect to frequency for a pair of wet electrodes;

FIG. 8C illustrates a graph depicting a variation of p-values of measurement of impedance magnitude with respect to frequency for a pair of wet electrodes;

FIGS. 9A and 9B illustrate graphs depicting a variation of impedance phase angle with respect to frequency for a pair of wet electrodes;

FIG. 9C illustrates a graph depicting a variation of percentage change of impedance phase angle with respect to frequency for a pair of wet electrodes;

FIG. 9D illustrates a graph depicting a variation of p-values of measurement of impedance phase angle with respect to frequency for a pair of wet electrodes;

FIG. 10 illustrates a graph depicting a variation of average percentage change of impedance magnitude with respect to volume of a fluid for a pair of wet electrodes;

FIGS. 11A and 11B illustrate graphs depicting a variation of impedance magnitude with respect to frequency for a pair of dry electrodes;

FIG. 11C illustrates a graph depicting a variation of percentage change of impedance magnitude with respect to frequency for a pair of dry electrodes;

FIG. 11D illustrates a graph depicting a variation of p-values of measurement of impedance magnitude with respect to frequency for a pair of dry electrodes;

FIGS. 12A and 12B illustrate graphs depicting a variation of impedance phase angle with respect to frequency for a pair of dry electrodes;

FIG. 12C illustrates a graph depicting a variation of percentage change of impedance phase angle with respect to frequency for a pair of dry electrodes;

FIG. 12D illustrates a graph depicting a variation of p-values of measurement of impedance phase angle with respect to frequency for a pair of dry electrodes;

FIG. 13 illustrates a graph depicting a variation of average percentage change of impedance magnitude with respect to volume of a fluid for a pair of dry electrodes;

FIG. 14 illustrates a graph depicting a variation of average percentage change of reactance with respect to volume of a fluid for a pair of dry electrodes;

FIG. 15 illustrates a graph depicting a variation of average percentage change of capacitance with respect to volume of a fluid for a pair of dry electrodes; and

FIG. 16 illustrates a graph comparing a percentage change of electrical characteristics measured using a pair of wet electrodes and a pair of dry electrodes.

### DETAILED DESCRIPTION

In the following description, reference is made to the accompanying figures that form a part thereof, and in which various embodiments are shown by way of illustration. It is to be understood that other embodiments are contemplated and may be made without departing from the scope or spirit of the present disclosure. The following detailed description, therefore, is not to be taken in a limiting sense.

As used herein, “at least one of A and B” should be understood to mean “only A, only B, or both A and B”.

An intravenous therapy may be performed on a patient to deliver fluids, such as medicines and nutrients, intravenously (i.e., directly into veins). The intravenous therapy may be performed using an intravenous catheter. The intravenous catheter may be supported or affixed at an injection site of the patient by an article (e.g., a securement film, a dressing, a tape, etc.).

In some cases, the intravenous catheter may need to be carefully and continuously administered by a medical practitioner, particularly for patients with fragile vasculature (e.g., infants and elderly). Extra-venous delivery or leakage of the fluids into a tissue at the injection site may occur due to misplacement of the intravenous catheter during initial insertion. Extra-venous delivery of the fluids into the tissue may also occur due to movement of the intravenous catheter during the intravenous therapy. Extra-venous delivery of the fluids may result in various complications, such as phlebitis, air embolism, hypervolemia, infection, and the like.

Thus, the medical practitioner may be required to periodically perform an assessment of the injection site. Examples of the assessment may include detecting softness, warmth, dryness, swelling, etc., of the injection site to detect extravasation. In other words, the assessment of the injection site may include manual inspection techniques that may be observational and subjective. Therefore, the assessment may not be accurate and reliable for detecting extravasation. In some cases, the assessment may only determine extravasation after a significant volume of the fluid is extravasated into the tissue.

Moreover, the assessment may be burdensome on the medical practitioner, as the assessment may have to be performed frequently in particular cases.

For example, the medical practitioner may be required to perform the assessment of the injection site every 4 hours in case of healthy and alert patients. In another example, the medical practitioner may be required to perform the assessment of the injection site every 1-2 hours in case of critically ill patients, aphasic patients, dysphasic patients, dysarthric patients, and patients with high-risk injection sites. In another example, the medical practitioner may be required to perform the assessment of the injection site every hour in case of neonatal and pediatric patients. In yet another example, the medical practitioner may be required to perform the assessment of the injection site every 5-10 minutes for administration of vesicants agents and vasoconstrictor agents. Therefore, the assessment of the injection site may be time consuming and may increase a workload of the medical practitioner.

Severe cases of extravasation that are not quickly detected may result in pain, tissue damage, and even death of the patient. Therefore, monitoring of extravasation during a course of the intravenous therapy may be vital. Moreover, upon the detection of extravasation, quantification of an extravasated volume of the fluid may be required to assess tissue damage at the injection site, and to provide appropriate treatment to the patient based on the extravasated volume of the fluid.

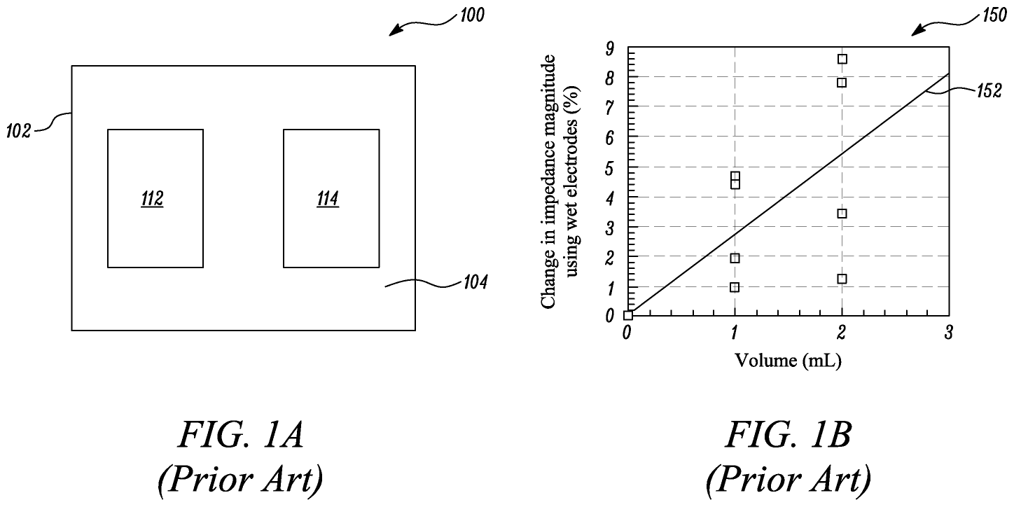

Referring now to Figures, FIG. 1A illustrates a schematic bottom view of a conventional sensor patch 100 for detecting extravasation into a tissue. The conventional sensor patch 100 includes a body 102 including a major surface 104. The conventional sensor patch 100 further includes a first wet electrode 112 disposed on the major surface 104. The conventional sensor patch 100 further includes a second wet electrode 114 disposed on the major surface 104 and spaced apart from the first wet electrode 112. The conventional sensor patch 100 may be placed on a skin of a patient, such that the first wet electrode 112 and the second wet electrode 114 contact the skin and flank a vein, into which a fluid (e.g., a medicine) is to be administered.

The skin of the patient proximal to an injection site may need to be hydrated by applying a suitable hydrogel on the skin during use of the conventional sensor patch 100. The hydrogel may electrically couple each of the first wet electrode 112 and the second wet electrode 114 to a tissue proximal to the vein. As used herein, the term “tissue” refers to a body tissue surrounding, or in a vicinity of a vein, into which a fluid may be administered during an intravenous therapy.

An input signal (e.g., an alternating current) having a predetermined frequency may be provided across the first wet electrode 112 and the second wet electrode 114. In response to the input signal, an output signal may be determined across the first wet electrode 112 and the second wet electrode 114. An impedance magnitude at the injection site may be determined based on the input signal and the output signal.

Prior to administering the fluid into the vein, a baseline impedance magnitude at the injection site may be determined based on the input signal and the output signal. The baseline impedance magnitude may be the impedance magnitude at the injection site prior to extravasation of the fluid into the tissue.

Thereafter, the fluid may be administered into the vein. In some cases, extravasation may take place, and the fluid may extravasate into the tissue. The extravasation of the fluid into the tissue causes a change in the impedance magnitude at the injection site from the baseline impedance magnitude. The conventional sensor patch 100 may be used to detect that the fluid has extravasated into the tissue based on the change in the impedance magnitude at the injection site from the baseline impedance magnitude.

An experiment was conducted to determine a sensitivity of the conventional sensor patch 100 to the change in the impedance magnitude at the injection site caused due to extravasation of small volumes of the fluid into the tissue. In the experiment, the input signal had a frequency of about 100 kilohertz.

The experiment was performed at four injection sites. A baseline impedance magnitude at each injection site of the four injection sites was determined. Subsequently, two milliliters (ml) of the fluid was intentionally injected into tissues at the four injection sites to simulate extravasation. The change in the impedance magnitude at the four injection sites was observed for each ml of the fluid injected into the tissues. Furthermore, a percentage change of the impedance magnitude from the corresponding baseline impedance magnitude was determined based on the change in the impedance magnitude due to injection of the fluid.

FIG. 1B illustrates a graph 150 depicting a percentage change of the impedance magnitude with respect to an extravasated volume of the fluid. The percentage change of the impedance magnitude is shown along the abscissa (Y-axis) and the extravasated volume of the fluid is shown along the ordinate (X-axis).

Referring to FIGS. 1A and 1B, for the input signal having a frequency of about 100 kilohertz, the first wet electrode 112 and the second wet electrode 114 provided a data set (shown by squares in FIG. 1B) correlating the percentage change of the impedance magnitude to the extravasated volume of the fluid.

A best linear fit to the data set is depicted by a line 152. As depicted by the line 152, the best linear fit had a slope of about 2.7% per milliliter of the fluid. Furthermore, as depicted by the line 152, the best linear fit had a coefficient of determination of about 0.75.

It was noted that the best linear fit had a low magnitude of the slope (i.e., about 2.7%) and a low coefficient of determination (i.e., about 0.75). Thus, it was concluded that the conventional sensor patch 100 had a low sensitivity to the change in the impedance magnitude at the four injection sites caused due to extravasation of small volumes of the fluid into the tissues.

The conventional sensor patch 100 may not be used to reliably detect extravasation of the small volumes of the fluid into the tissue. Further, the conventional sensor patch 100 may not be suitable for quantifying the extravasated volume of the fluid into the tissue due to the low sensitivity to the change in the impedance magnitude.

The conventional sensor patch 100 may have additional drawbacks. For example, skin preparation (i.e., shaving and scrubbing the skin with an abrasive) may be required before use of the conventional sensor patch 100. Further, the skin of the patient proximal to the injection site may need to be hydrated by the suitable hydrogel to electrically couple each of the first and second wet electrodes 112, 114 to the tissue. Therefore, the impedance magnitude determined using the conventional sensor patch 100 may be dependent on the hydration level of the tissue.

Since hydration of the tissue may take time, initial tissue impedance measurements determined using the conventional sensor patch 100 may drift as the tissue moistens. Further, since the hydration level of the tissue may also vary with time, the tissue impedance measurements may drift over time. The drift of the tissue impedance measurements may complicate algorithms for detecting extravasation. The drift of the tissue impedance measurements may also introduce inconsistencies in the tissue impedance measurements, and thus may result in unreliable detection of extravasation using the conventional sensor patch 100.

Furthermore, wet electrodes (e.g., the first and second wet electrodes 112, 114) may dry out over time. Therefore, the first and second wet electrodes 112, 114 may be electrically coupled to the tissue up to a hydration time provided by the hydrogel. In some cases, the hydration time may be less than a course of an intravenous therapy.

Moreover, a stratum corneum of the skin may exhibit strong impedance that may mask small changes in the impedance magnitude at the injection sites. Thus, the small changes in the impedance magnitude at the injection sites may not be detectable using the conventional sensor patch 100.

Therefore, the conventional sensor patch 100 may not be sensitive to the small changes in the impedance magnitude at the injection sites. Consequently, the conventional sensor patch 100 may not be used to reliably detect extravasation of the small volumes of the fluid into the tissue. Further, the conventional sensor patch 100 may not be used to quantify the extravasated volume of the fluid into the tissue.

The present disclosure provides an article for detecting extravasation into a tissue. The article includes a body. The boy includes a first major surface, an opposing second major surface, a first side, and an opposing second side. The article further includes a first electrode disposed on the first major surface of the body. The first electrode includes at least one skin-penetrating microfeature. The article further includes a second electrode disposed on the first major surface of the body. The second electrode includes at least one skin-penetrating microfeature. The first electrode is electrically connected to the second electrode.

The article of the present disclosure may be used for detecting and quantifying extravasation of small volumes of a fluid into the tissue. Specifically, the skin-penetrating microfeatures of the first and second electrodes may provide improved sensitivity to small changes in impedance at the injection site caused due to extravasation of the fluid into the tissue. Therefore, the article may allow quicker detection of extravasation and quantification of the extravasated volume of the fluid into the tissue.

The skin-penetrating microfeatures may penetrate and bypass the stratum corneum. Thus, little to no skin preparation (i.e., shaving and scrubbing the skin with an abrasive) may be required before use of the article, as compared to the conventional sensor patch 100. Further, as the stratum corneum of the skin exhibits strong impedance, bypassing the stratum corneum may provide improved sensitivity to small changes in impedance at the injection sites caused due to extravasation.

The skin-penetrating microfeatures may further provide a stable electrical interface with the tissue. The electrical interface between each of the first and second electrodes and the tissue may not be dependent on hydration level of the tissue. Thus, the article of the present disclosure may be used to detect extravasation for a longer duration, as compared to the conventional sensor patch 100.

In some cases, the article of the present disclosure may be used to generate an alert or an alarm to notify a medical practitioner upon detecting extravasation. Further, the article may communicate information related to extravasation with other systems, such as monitoring systems. Thus, the article may allow the medical practitioner to assess tissue damage at the injection site and provide appropriate treatment to the patient. The article may also be used to continuously monitor extravasation and accurately detect extravasation. The article may also reduce a workload of the medical practitioner, as the medical practitioner may not need to periodically perform assessment of the injection site.

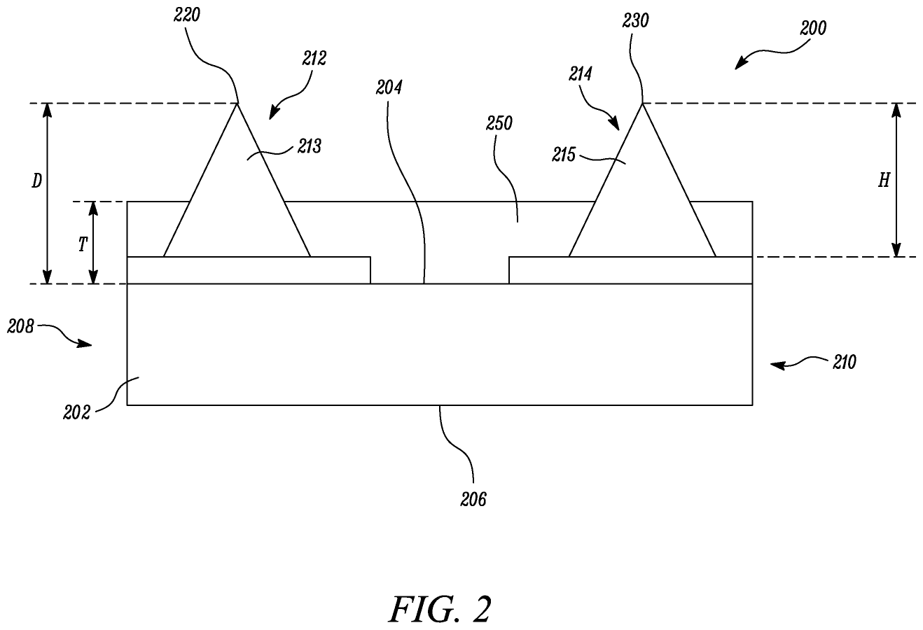

FIG. 2 illustrates a sectional side view of an article 200 for detecting extravasation into a tissue according to an embodiment of the present disclosure. The article 200 may be used for detecting extravasation into the tissue by monitoring at least one electrical parameter (e.g., impedance magnitude, impedance phase angle, capacitance, etc.).

The article 200 includes a body 202. The body 202 includes a first major surface 204, an opposing second major surface 206, a first side 208, and an opposing second side 210.

In some embodiments, the body 202 includes a polymer film. In some embodiments, the polymer film may include one or more polymers, such as cellulose acetate butyrate, cellulose acetate propionate, cellulose triacetate, poly(meth)acrylates such as polymethyl methacrylate, polyesters such as polyethylene terephthalate and polyethylene naphthalate, copolymers or blends based on naphthalene dicarboxylic acids, polyether sulfones, polyurethanes, polycarbonates, polyvinyl chloride, syndiotactic polystyrene, cyclic olefin copolymers, and polyolefins including polyethylene and polypropylene such as cast and biaxially oriented polypropylene. In some embodiments, the polymer film may include a single layer, or multiple layers such as polyethylene-coated polyethylene terephthalate.

The article 200 further includes a first electrode 212 and a second electrode 214. The first electrode 212 is electrically connected to the second electrode 214.

The first electrode 212 is disposed on the first major surface 204 of the body 202. In the illustrated embodiment of FIG. 2, the first electrode 212 is disposed on the first major surface 204 proximal to the first side 208 of the body 202. The first electrode 212 includes at least one skin-penetrating microfeature 213. The at least one skin-penetrating microfeature 213 of the first electrode 212 may include a microneedle, or similar small, pointed structure configured to at least partially penetrate a skin of a patient.

The second electrode 214 is disposed on the first major surface 204 of the body 202. In the illustrated embodiment of FIG. 2, the second electrode 214 is disposed on the first major surface 204 proximal to the second side 210 of the body 202. In other words, in the illustrated embodiment of FIG. 2, the second electrode 214 is spaced apart from the first electrode 212. The second electrode 214 includes at least one skin-penetrating microfeature 215. The at least one skin-penetrating microfeature 215 of the second electrode 214 may include a microneedle, or similar small, pointed structure configured to penetrate the skin. The first electrode 212 and the second electrode 214 may be interchangeably referred to as “dry electrodes”.

In some embodiments, the at least one skin-penetrating microfeature of each of the first electrode 212 and the second electrode 214 is electrically conductive. In the illustrated embodiment of FIG. 2, the at least one skin-penetrating microfeature 213 of the first electrode 212 is electrically conductive. Further, in the illustrated embodiment of FIG. 2, the at least one skin-penetrating microfeature 215 of the second electrode 214 is electrically conductive.

In some embodiments, the at least one skin-penetrating microfeature of at least one of the first electrode 212 and the second electrode 214 includes at least one of a silver coated microparticle and a silver chloride coated microparticle. In some embodiments, the at least one skin-penetrating microfeature 213 of the first electrode 212 may be a silver coated microparticle or a silver chloride coated microparticle. Further, in some embodiments, the at least one skin-penetrating microfeature 215 of the second electrode 214 may be a silver coated microparticle or a silver chloride coated microparticle.

In some embodiments, the at least one skin-penetrating microfeature 213 of the first electrode 212 may be a silver coated microparticle and the at least one skin-penetrating microfeature 215 of the second electrode 214 may be a silver chloride coated microparticle. Silver and silver chloride may allow the following reversible reactions to take place, and thus may allow detecting the at least one electrical parameter.

$${Ag}^{+} + {e}^{-} ⇌ Ag ( s ) \\ AgCl ( s ) + {e}^{-} ⇌ Ag ( s ) + {Cl}^{-}$$

In some embodiments, the at least one skin-penetrating microfeature of at least one of the first electrode 212 and the second electrode 214 further includes a point. In the illustrated embodiment of FIG. 2, the at least one skin-penetrating microfeature 213 of the first electrode 212 includes a point 220. Moreover, in the illustrated embodiment of FIG. 2, the at least one skin-penetrating microfeature 215 of the second electrode 214 further includes a point 230. The point 220 of the at least one skin-penetrating microfeature 213 and the point 230 of the at least one skin-penetrating microfeature 215 may facilitate penetration of the skin.

In the illustrated embodiment of FIG. 2, the at least one skin-penetrating microfeature 213 of the first electrode 212 defines a distance D between the first major surface 204 and the point 220. In some embodiments, the point 220 extends from the first major surface 204 by about 50 micrometers to about 1000 micrometers. In other words, in some embodiments, the distance D is from about of 50 micrometers to about 1000 micrometers. In some embodiments, the point 230 of the at least one skin-penetrating microfeature 215 of the second electrode 214 extends from the first major surface 204 by about 50 micrometers to about 1000 micrometers. In other words, in some embodiments, the point 230 of the at least one skin-penetrating microfeature 215 of the second electrode 214 may define the distance D similar to the at least one skin-penetrating microfeature 213 of the first electrode 212.

In some embodiments, the at least one skin-penetrating microfeature of at least one of the first electrode 212 and the second electrode 214 has at least one dimension ranging from about 175 micrometers to about 1500 micrometers. In other words, in some embodiments, the at least one skin-penetrating microfeature of at least one of the first electrode 212 and the second electrode 214 has at least one of a length, a width, and a height ranging from about 175 micrometers to about 1500 micrometers. In the illustrated embodiment of FIG. 2, the at least one skin-penetrating microfeature 215 of the second electrode 214 has a height H. Further, in some embodiments, the height H of the at least one skin-penetrating microfeature 215 is from about 175 micrometers to about 1500 micrometers. In some embodiments, the height H of the at least one skin-penetrating microfeature 215 is about 400 micrometers.

Consequently, in some embodiments, the at least one skin-penetrating microfeature of at least one of the first electrode 212 and the second electrode 214 is configured to fully penetrate a stratum corneum and partially penetrate an epidermis. In the illustrated embodiment of FIG. 2, each of the at least one skin-penetrating microfeature 213 of the first electrode 212 and the at least one skin-penetrating microfeature 215 of the second electrode 214 is configured to fully penetrate the stratum corneum and partially penetrate the epidermis. In some cases, fully penetrating the stratum corneum and partially penetrating the epidermis may improve detection of changes in the at least one electrical parameter.

Moreover, in some embodiments, the at least one skin-penetrating microfeature of at least one of the first electrode 212 and the second electrode 214 provides a stable electrical interface between the tissue and each of the first electrode 212 and the second electrode 214. In the illustrated embodiment of FIG. 2, the at least one skin-penetrating microfeature 213 of the first electrode 212 and the at least one skin-penetrating microfeature 215 of the second electrode 214 provide the stable electrical interface between the tissue and the corresponding first and second electrodes 212, 214.

The stable electrical interface may be provided by the first electrode 212 and the second electrode 214 without an application of a hydrogel on the skin. Thus, the stable electrical interface may not degrade with time, in contrast to electrodes that may require the application of the hydrogel on the skin (i.e., wet electrodes). Therefore, the stable electrical interface between the tissue and each of the first electrode 212 and the second electrode 214 may allow reliable and consistent determination of an accurate value of the at least one electrical property throughout a course of an intravenous therapy using the article 200.

In some embodiments, the body 202 further includes an adhesive layer 250 disposed on at least a portion of the first major surface 204 of the body 202. In some embodiments, the adhesive layer 250 may be disposed on at least 30%, at least 40%, at least 50%, at least 60%, at least 70%, at least 80%, or at least 90% of the first major surface 204. In some embodiments, a thickness T of the adhesive layer 250 may range from about 150 micrometers to about 200 micrometers. In some other embodiments, the thickness T of the adhesive layer 250 may range from about 200 micrometers to about 1200 micrometers. In some embodiments, the thickness T of the adhesive layer 250 may be non-uniform, and vary between about 150 micrometers and about 1200 micrometers.

In some embodiments, the adhesive layer 250 is configured to detachably attach the body 202 to the skin. The adhesive layer 250 may include any medically-acceptable adhesive. The adhesive layer 250 may include, for example, an acrylic adhesive, a rubber adhesive, a high-tack silicone adhesive, a polyurethane adhesive, and the like. In some embodiments, the adhesive layer 250 may include a pressure-sensitive adhesive. In some embodiments, the adhesive layer 250 may further include anti-microbial agents to reduce microbial activity on the skin.

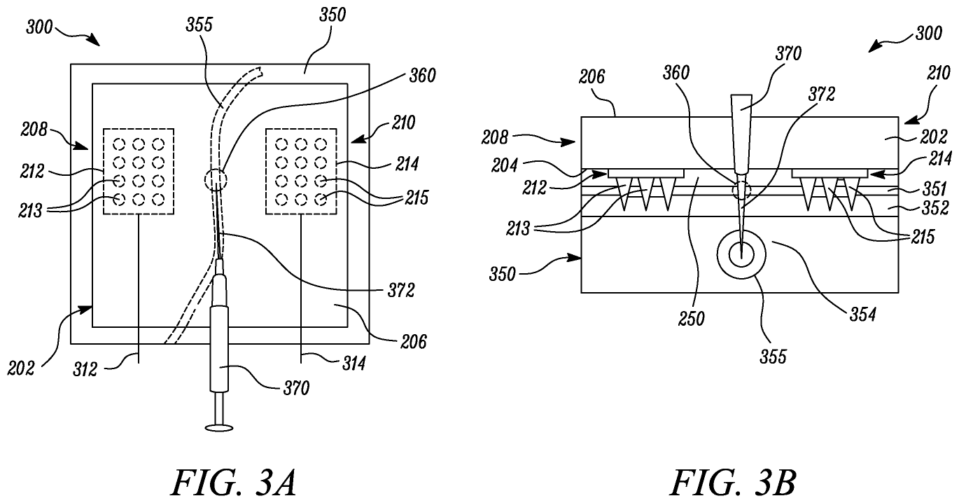

FIGS. 3A and 3B illustrate a schematic top view and a sectional side view, respectively, of an article 300 according to another embodiment of the present disclosure. The article 300 is similar to the article 200 of FIG. 2, with like elements designated by like numerals. However, the article 300 has a different configuration of the first electrode 212 and the second electrode 214 as compared to the article 200. Some elements of the article 300 are not shown in FIGS. 3A and 3B for illustrative purposes.

In the illustrated embodiment of FIGS. 3A and 3B, the body 202 has a substantially rectangular shape. However, the body 202 may have any suitable shape, such as curved, triangular, polygonal, circular, elliptical, and so forth, based on desired application attributes.

In FIGS. 3A and 3B, the article 300 is placed on a skin 350 of a patient. Specifically, in FIGS. 3A and 3B, the article 300 is placed on the skin 350 at an injection site 360 (shown by circles in FIGS. 3A and 3B). The injection site 360 may be proximal to, or directly above a vein 355 into which a fluid (e.g., a medicine) may be administered. A tissue 354 surrounds the vein 355. In some cases, extravasation may occur, and the fluid may extravasate into the tissue 354 from the vein 355.

In some embodiments, the at least one skin-penetrating microfeature of at least one of the first electrode 212 and the second electrode 214 includes more than one skin-penetrating microfeatures. In other words, in some embodiments, the at least one skin-penetrating microfeature 213 of the first electrode 212 may include a plurality of microneedles, or similar small, pointed structures to at least partially penetrate the skin 350. Further, in some embodiments, the at least one skin-penetrating microfeature 215 of the second electrode 214 may include a plurality of microneedles, or similar small, pointed structures to at least partially penetrate the skin 350.

Specifically, in the illustrated embodiment of FIGS. 3A and 3B, the at least one skin-penetrating microfeature 213 of the first electrode 212 includes more than one skin-penetrating microfeatures 213. Moreover, in the illustrated embodiment of FIGS. 3A and 3B, the at least one skin-penetrating microfeature 215 of the second electrode 214 includes more than one skin-penetrating microfeatures 215.

The more than one skin-penetrating microfeatures 213 of the first electrode 212 and the more than one skin-penetrating microfeatures 215 of the second electrode 214 may improve an electrical coupling of the first electrode 212 and the second electrode 214 with the tissue 354. Further, the more than one skin-penetrating microfeatures 213 of the first electrode 212 and the more than one skin-penetrating microfeatures 215 of the second electrode 214 may facilitate in providing a stable electrical interface between the tissue 354 and each of the first electrode 212 and the second electrode 214.

As discussed above, in FIGS. 3A and 3B, the article 300 is placed on the skin 350 at the injection site 360. Specifically, in some embodiments, the first major surface 204 of the body 202 is configured to be placed on the injection site 360, such that the injection site 360 is disposed between the first side 208 and the second side 210 of the body 202. Moreover, in the illustrated embodiment of FIGS. 3A and 3B, the first major surface 204 of the body 202 is configured to be placed on the injection site 360, such that the injection site 360 is disposed between the first electrode 212 and the second electrode 214. Consequently, in the illustrated embodiment of FIGS. 3A and 3B, the first electrode 212 and the second electrode 214 flank the vein 355.

As shown in FIG. 3B, the skin 350 includes a stratum corneum 351 and an epidermis 352. In some embodiments, the at least one skin-penetrating microfeature of at least one of the first electrode 212 and the second electrode 214 is configured to fully penetrate the stratum corneum 351 and partially penetrate the epidermis 352.

In the illustrated embodiment of FIGS. 3A and 3B, the more than one skin-penetrating microfeatures of 213 of the first electrode 212 are configured to fully penetrate the stratum corneum 351 and partially penetrate the epidermis 352. Further, in the illustrated embodiment of FIGS. 3A and 3B, the more than one skin-penetrating microfeatures of 215 of the second electrode 214 are configured to fully penetrate the stratum corneum 351 and partially penetrate the epidermis 352. In some cases, fully penetrating the stratum corneum 351 and partially penetrating the epidermis 352 may improve sensitivity to changes in the at least one electrical parameter.

In some embodiments, the article 300 further includes a means for detachably attaching the article 300 to the tissue 354. In the illustrated embodiment of FIG. 3B, the adhesive layer 250 is configured to detachably attach the body 202 to the skin 350. Thus, the adhesive layer 250 may detachably attach the article 300 to the tissue 354. In other words, in some embodiments, the means may include the adhesive layer 250 for detachably attaching the article 300 to the tissue 354. The means for detachably attaching the article 300 to the tissue 354 may further include, but is not limited to, a hook and loop material, such as VELCRO®, a support belt, a support strap, an adhesive tape, a bandage, a medical drape, a gauze roll, and a surgical tape.

As shown in FIGS. 3B, a cannula 372 of an intravenous catheter 370 may be inserted through the skin 350 until the cannula 372 enters the vein 355. The intravenous catheter 370 may be used to administer the fluid into the vein 355 through the cannula 372.

As discussed above, in some cases, the fluid may extravasate into the tissue 354 from the vein 355.

Upon extravasation of the fluid into the tissue 354, the at least one electrical parameter at the injection site 360 changes. The change in the at least one electrical parameter may be determined using the article 300, and thus may be used to detect extravasation.

As shown in FIG. 3A, in some embodiments, the article 300 further includes a first lead 312 electrically connected to the first electrode 212 and a second lead 314 electrically connected to the second electrode 214. The first and second leads 312, 314 may be configured to provide and determine electrical signals across the first electrode 212 and the second electrode 214. In some embodiments, the first and second leads 312, 314 may include electrical wires. The electrical wires may include any suitable conductor, such as, copper, aluminum, silver, and the like. In some other embodiments, the first and second leads 312, 314 may include a conductive ink, such as silver ink, that may be 3D printed, screen-printed, vapor-deposited, or transferred on the body 202.

In some embodiments, a controller (not shown in FIGS. 3A and 3B) may be electrically connected to the first electrode 212 by the first lead 312 and the second electrode 214 by the second lead 314. The controller may be configured to provide electrical signals to the first electrode 212 and receive electrical signals from the second electrode 214. In some embodiments, the controller may be disposed external to the body 202 of the article 300. In some other embodiments, the controller may be disposed on the body 202.

The controller may determine a change in the at least one electrical parameter upon extravasation based on the electrical signals provided to the first electrode, and received from the second electrode. Further, the controller may detect extravasation based on the change in the at least one electrical parameter.

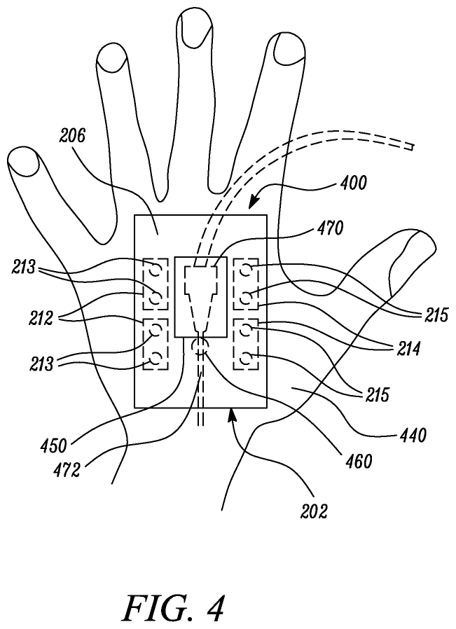

FIG. 4 illustrates a schematic top view of an article 400 according to another embodiment of the present disclosure. The article 400 is similar to the article 300 of FIGS. 3A and 3B, with like elements designated by like numerals. However, the article 400 has a different configuration of the first electrode 212 and the second electrode 214 as compared to the article 300. Further, the article 400 includes additional elements as compared to the article 300. Some elements of the article 400 are not shown in FIG. 4 for illustrative purposes.

In FIG. 4, the article 400 is placed on a skin 440 at an injection site 460 (shown by a circle in FIG. 4).

In the illustrated embodiment of FIG. 4, the article 400 includes a plurality of the first electrodes 212. In the illustrated embodiment of FIG. 4, the article 400 further includes a plurality of the second electrodes 214. Specifically, in the illustrated embodiment of FIG. 4, the plurality of the first electrodes 212 and the plurality of the second electrodes 214 form an electrode array. The electrode array may be used to determine a spatial extent of extravasation proximal to the injection site 460 using the article 400. Further, the electrode army may reduce determination of anomalous values using the article 400, and thus reduce false positive detection of extravasation.

In the illustrated embodiment of FIG. 4, the article 400 further includes a securement device 450 disposed on the second major surface 206 of the body 202. In some embodiments, the securement device 450 may be adhered to the second major surface 206 by an adhesive. In some embodiments, the securement device 450 may be integral with the body 202 of the article 400.

In some embodiments, the securement device 450 is configured to detachably couple with an intravenous catheter 470. In the illustrated embodiment of FIG. 4, the securement device 450 has dimensions corresponding to the intravenous catheter 470 to detachably couple with the intravenous catheter 470. However, the dimensions of the securement device 450 may vary based on different types of the intravenous catheter 470. Moreover, the securement device 450 may include retention features (not shown) to secure the intravenous catheter 470.

Therefore, the securement device 450 may support and secure the intravenous catheter 470 at the injection site 460, and reduce movement of the intravenous catheter 470 during use. Specifically, the securement device 450 may reduce movement of a cannula 472 of the intravenous catheter 470 during use. In some cases, the securement device 450 may reduce an occurrence of extravasation due to movement of the intravenous catheter 470 during use.

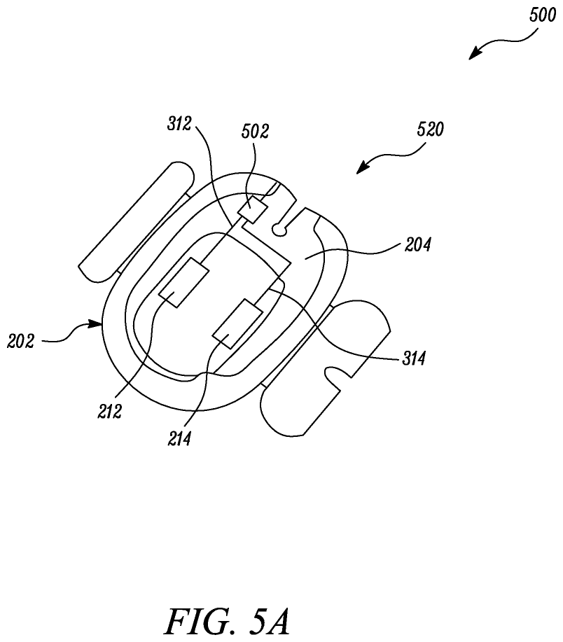

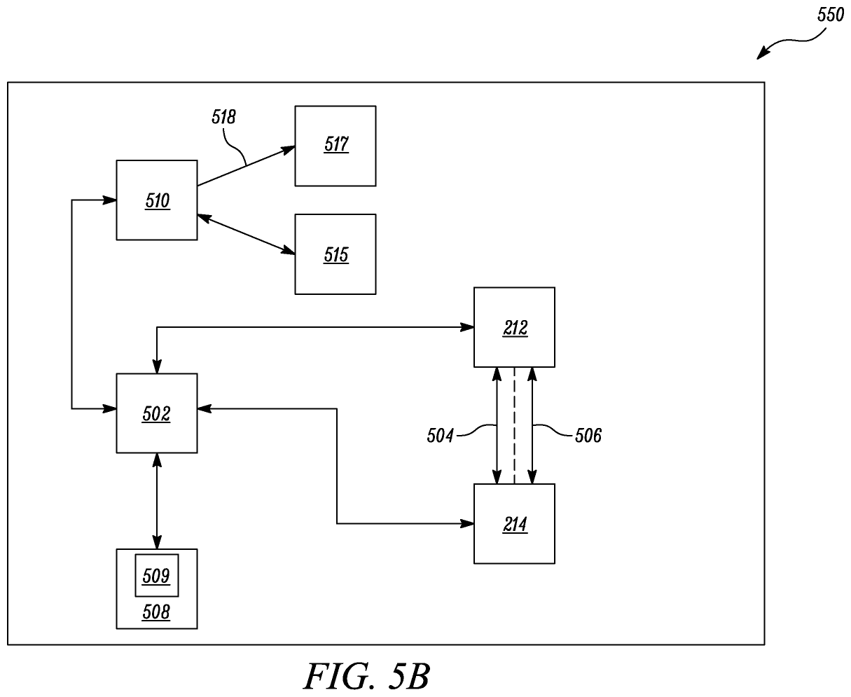

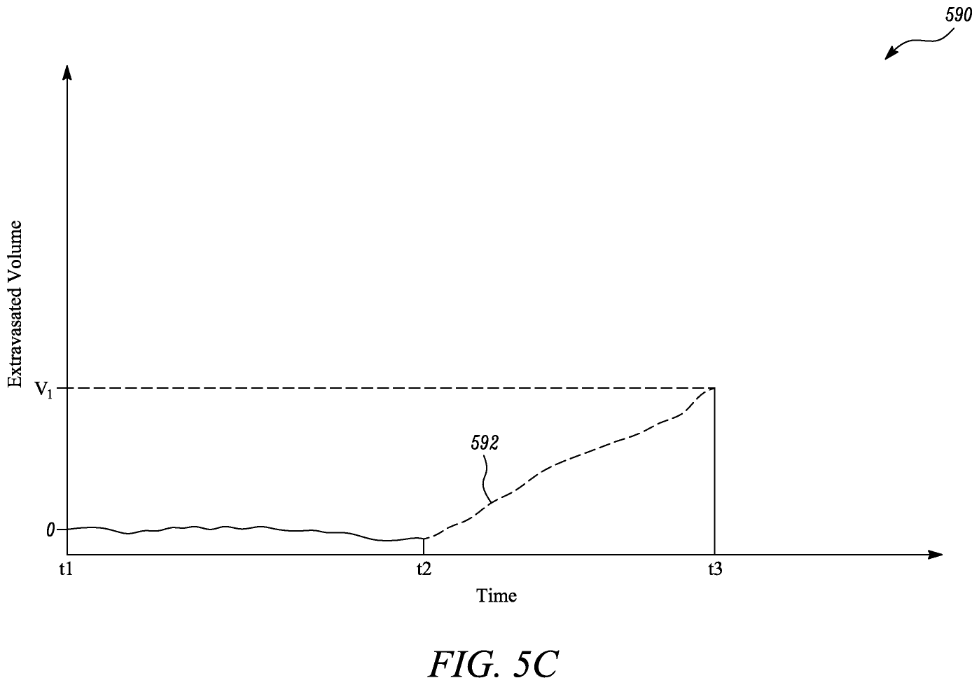

FIG. 5A illustrates a schematic bottom perspective view of a system 500 for detecting extravasation into a tissue according to an embodiment of the present disclosure.

In the illustrated embodiment of FIG. 5A, the system 500 includes an article 520. The article 520 is similar to the article 300 of FIGS. 3A and 3B, with like elements designated by like numbers. However, the body 202 of the article 520 has a different shape as compared to the article 300. Some elements of the article 520 are not shown in FIG. 5A for illustrative purposes. The article 520 includes the first electrode 212 disposed on the first major surface 204 of the body 202 and the second electrode 214 disposed on the first major surface 204 of the body 202.

The system 500 further includes a controller 502 electrically connected to the first electrode 212 and the second electrode 214. In the illustrated embodiment of FIG. 5A, the controller 502 is electrically connected to the first electrode 212 by the first lead 312 and to the second electrode 214 by the second lead 314.

As shown in FIG. 5A, in some embodiments, the controller 502 is coupled to the article 520. Specifically, in some embodiments, the controller 502 is coupled to the body 202 of the article 520. In the illustrated embodiment of FIG. 5A, the controller 502 is coupled to the first major surface 204 of the body 202. However, in some embodiments, the controller 502 may be coupled to the second major surface 206 of the body 202. In some other embodiments, the controller 502 is spaced apart from the article 520. In other words, in some other embodiments, the controller 502 may be an external controller disposed external to the article 520.

FIG. 5B illustrates a schematic block diagram of a system 550 according to an embodiment of the present disclosure. The system 550 is similar to the system 500 of FIG. 5A, with like elements designated by like numbers. However, the system 550 has additional elements as compared to the system 500.

Referring to FIG. 5B, the controller 502 is configured to provide an input signal 504 (e.g., an alternating current voltage, a direct current voltage, and the like) across the first electrode 212 and the second electrode 214. In some embodiments, the input signal 504 has a frequency ranging from about 0 hertz to about 1 gigahertz. In some embodiments, the input signal 504 has a frequency of about 1 megahertz. In some embodiments, the controller 502 may be configured to provide the input signal 504 across the first electrode 212 and the second electrode 214 via the first and second leads 312, 314 (shown in FIG. 5A).

The controller 502 is further configured to determine an output signal 506 (e.g., an alternating current, a direct current, and the like) across the first electrode 212 and the second electrode 214 in response to the input signal 504. In some embodiments, the controller 502 may be configured to determine the output signal 506 across the first electrode 212 and the second electrode 214 via the first and second leads 312, 314 (shown in FIG. 5A).

The controller 502 is further configured to determine at least one electrical parameter based on the output signal 506 and the input signal 504. In some embodiments, the at least one electrical parameter includes one or more of an impedance magnitude, an impedance phase angle, a capacitance, a resistance, and a reactance. In some cases, the at least one electrical parameter may be of a tissue (e.g., the tissue 354 shown in FIG. 3B).

The controller 502 is further configured to detect extravasation of a fluid into the tissue based on a change in the at least one electrical parameter. For example, in some cases, the fluid may be an ionic fluid (i.e., a conductive fluid), which may reduce the impedance magnitude at an injection site upon extravasation. In such cases, the controller 502 may detect extravasation based on a reduction in the impedance magnitude at the injection site. In some other cases, the fluid may be a non-ionic fluid (i.e., a non-conductive fluid), which may increase the impedance magnitude at the injection site upon extravasation. In such cases, the controller 502 may detect extravasation based on an increase in the impedance magnitude at the injection site.

In some embodiments, the controller 502 is further configured to determine at least one of an extravasated volume of the fluid and a time period of extravasation based on the change in the at least one electrical parameter. The controller 502 may utilize suitable algorithms to detect extravasation, determine the extravasated volume of the fluid, and determine the time period of extravasation based on the change in the at least one electrical parameter.

In the illustrated embodiment of FIG. 5B, the system 550 further includes a memory 508 communicably coupled to the controller 502.

The controller 502 may include one or more processors configured to perform the functions described herein. In addition to the one or more processors, the controller 502 may include integrated circuitry for processing and/or control, e.g., one or more processors and/or processor cores and/or Field Programmable Gate Array (FPGAs) and/or Application Specific Integrated Circuitry (ASICs). The controller 502 may be configured to access (e.g., write to and/or reading from) the memory 508, which may include any kind of volatile and/or non-volatile memory, e.g., cache and/or buffer memory and/or Random Access Memory (RAM) and/or Read-Only Memory (ROM) and/or optical memory and/or Erasable Programmable Read-Only Memory (EPROM). The memory 508 may be configured to store various algorithms and code executable by the controller 502 that may cause the one or more processors to perform the functions described herein.

In some embodiments, the controller 502 is further configured to store a timestamp 509 indicative of extravasation of the fluid in the memory 508. The timestamp 509 may include information related to a time of detection of extravasation of the fluid into the tissue.

In some embodiments, upon detecting extravasation of the fluid, the controller 502 is further configured to perform at least one of generating an alert; and providing a stop signal 518 to an infusion system 517 to automatically stop infusion of the fluid. The alert may include, for example, an audio alert, a visual alert, and the like. In some embodiments, the article 520 may further include light emitting diodes (LEDs) (not shown) to visually provide the visual alert generated by the controller 502. In one example, the LEDs may be configured to emit green light prior to detecting extravasation, and flash red light upon detecting extravasation. In some embodiments, the article 520 may further include a speaker (not shown) to audibly provide the audio alert generated by the controller 502. In one example, the speaker may be configured to beep upon detecting extravasation.

The alert generated by the controller 502 upon detecting extravasation of the fluid may notify the medical practitioner about extravasation. Thus, the medical practitioner may stop the intravenous therapy in a timely manner, and reduce potential complications that may arise due to prolonged extravasation.

In the illustrated embodiment of FIG. 5B, the system 550 further includes a communication module 510 communicably coupled to the controller 502. The communication module 510 may allow wired and/or wireless communication between the controller 502 and various other systems. The communication module 510 may use any suitable communication protocol and technology for communication between the controller 502 and the other systems. For example, the communication module 510 may utilize Transmission Control Protocol/Internet Protocol (TCP/IP), Bluetooth®, WiFi, WiMax, cellular technologies, e.g., Long Term Evolution (LTE), Near-Field Communication (NFC), and/or Far-Field Circuitry associated with RF-ID protocols.

In the illustrated embodiment of FIG. 5B, the system 550 further includes one or more monitoring devices 515. Further, in the illustrated embodiment of FIG. 5B, the controller 502 is configured to transmit information to the one or more monitoring devices 515 via the communication module 510. In some cases, the controller 502 may receive information from the one or more monitoring devices 515 via the communication module 510. Examples of the one or more monitoring devices may include smartphones, tablets, computers, wireless headsets, and the like. In some embodiments, the controller 502 may be configured to continuously communicate information to the one or more monitoring devices 515 via the communication module 510. In some embodiments, the one or more monitoring devices 515 may be configured to represent the information received from the controller 502 via a user interface.

In the illustrated embodiment of FIG. 5B, the system 550 further includes the infusion system 517. The infusion system 517 may be configured to intravenously administer the fluid to the patient. Furthermore, in the illustrated embodiment of FIG. 5B, the controller 502 is configured to transmit information and signals to the infusion system 517 via the communication module 510.

FIG. 5C illustrates the information displayed by the one or more monitoring devices 515 (shown in FIG. 5B) according to an embodiment of the present disclosure. As shown in FIG. 5C, in some embodiments, the one or more monitoring devices 515 are configured to process and graphically represent the information received from the controller 502 (shown in FIG. 5B) as a graph 590 on a display. In such embodiments, the one or more monitoring devices 515 may display the information received from the controller 502 continuously on a cathode ray oscilloscope (CRO), a cathode ray tube (CRT), a Liquid Crystal Display (LCD), and the like, as data channels along a time axis.

The graph 590 depicts a variation of the extravasated volume of the fluid with respect to time. The extravasated volume of the fluid is shown along the abscissa (Y-axis) and time is shown along the ordinate (X-axis).

Referring to FIGS. 5B and 5C, the graph 590 includes a curve 592 corresponding to the information received from the controller 502 and displayed by the one or more monitoring devices 515 in real-time.

At a first time t1, the one or more monitoring devices 515 starts to receive the information from the controller 502 and displays the curve 592 corresponding to the information received from the controller 502.

At a second time t2, the controller 502 detects extravasation. As discussed above, in some embodiments, the controller 502 is configured to store the timestamp 509 indicative of extravasation of the fluid in the memory 508. In this case, the timestamp 509 may be indicative of the second time t2. In some cases, the one or more monitoring devices 515 may be configured to receive the alert generated by the controller 502 upon detecting extravasation (i.e., at the second time t2) and generate an alarm to notify the medical practitioner. The alarm may include an audio alarm, a visual alarm, and the like.

At a third time t3, the medical practitioner may stop the intravenous therapy after being alerted of extravasation. As discussed above, in some embodiments, upon detecting extravasation of the fluid, the controller 502 is further configured to perform providing the stop signal 518 to the infusion system 517 to automatically stop infusion of the fluid. The medical practitioner may identify the extravasated volume of the fluid based on the curve 592. As shown in FIG. 5C, the medical practitioner may identify that the extravasated volume is about a volume V1 at the third time t3. Therefore, the medical practitioner may be able to assess tissue damage at an injection site, and provide appropriate treatment to the patient based on the volume V1.

Further, the medical practitioner may identify the time period of extravasation based on the curve 592. In this case, the time period of extravasation is a time period between the third time t3 and the second time t2 (i.e., t3-t2).

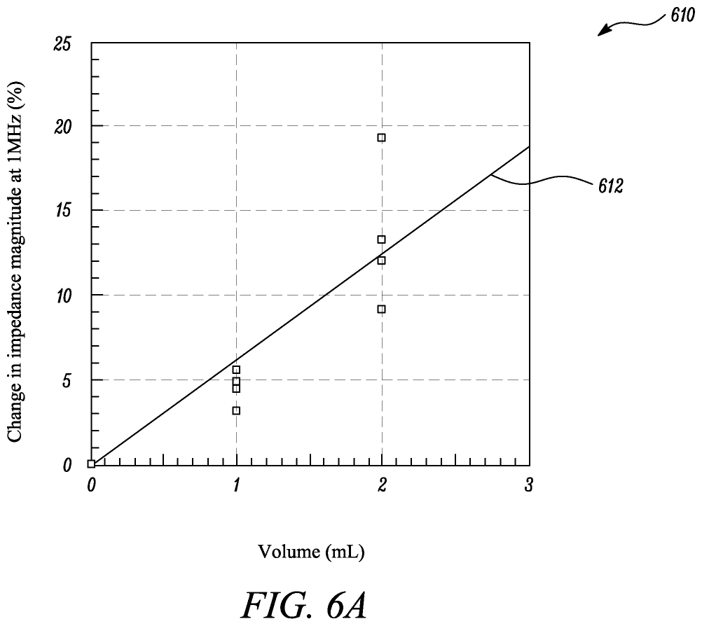

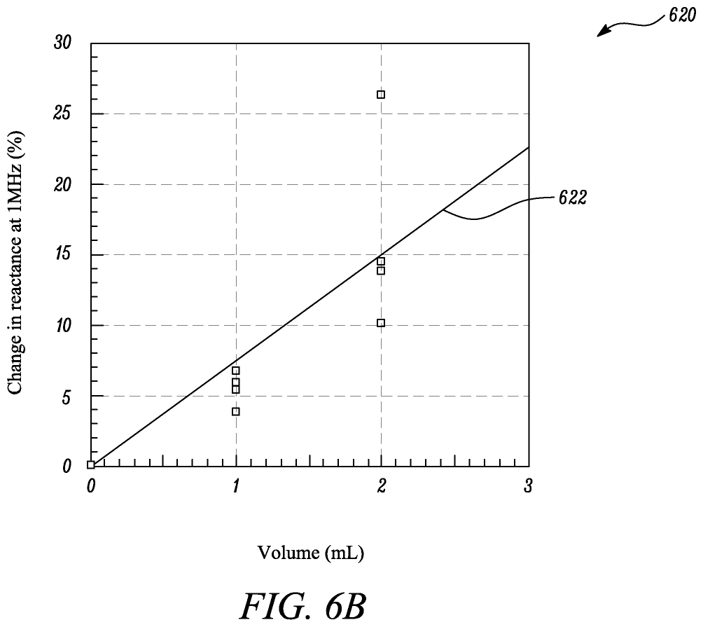

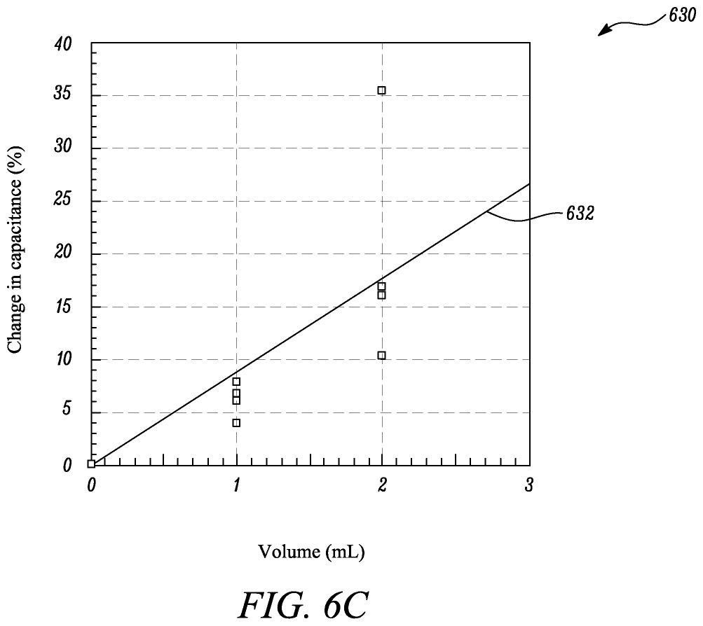

FIGS. 6A-6C illustrate graphs 610, 620, 630 depicting a percentage change of the at least one electrical parameter with respect to an extravasated volume of the fluid. The percentage change of at least one electrical parameter is shown along the abscissa (Y-axis) and the extravasated volume of the fluid in milliliters is shown along the ordinate (X-axis).

Referring to FIGS. 5B and 6A, in some embodiments, for the input signal 504 having a frequency of about 1 megahertz, the first electrode 212 and the second electrode 214 provide a data set (shown by squares in FIG. 6A) correlating the percentage change of the at least one electrical parameter to the extravasated volume of the fluid.

In the graph 610, the at least one electrical parameter is the impedance magnitude. Specifically, in some embodiments, for the input signal 504 having a frequency of about 1 megahertz, the first electrode 212 and the second electrode 214 provide the data set correlating a percentage change of the impedance magnitude to the extravasated volume of the fluid.

Further, in the graph 610, a best linear fit to the data set is depicted by a line 612. In some embodiments, the best linear fit to the data set has a slope of at least about 5% per unit volume of the fluid. Specifically, the best linear fit to the data set has a slope of at least about 5% per milliliter of the fluid.

As depicted by the line 612, the best linear fit to the data set has a slope of about 6.2% per milliliter of the fluid. Further, in some embodiments, the best linear fit has a coefficient of determination of at least about 0.8. In some embodiments, the best linear fit has a coefficient of determination of about 0.9. As depicted by the line 612, the best linear fit has a coefficient of determination of about 0.91.

Referring to FIGS. 5B and 6B, in some embodiments, for the input signal 504 having a frequency of about 1 megahertz, the first electrode 212 and the second electrode 214 provide a data set (shown by squares in FIG. 6B) correlating the percentage change of the at least one electrical parameter to the extravasated volume of the fluid.

In the graph 620, the at least one electrical parameter is the reactance. Specifically, in some embodiments, for the input signal 504 having a frequency of about 1 megahertz, the first electrode 212 and the second electrode 214 provide the data set correlating a percentage change of the reactance to the extravasated volume of the fluid.

Further, in the graph 620, a best linear fit to the data set is depicted by a line 622. In some embodiments, the best linear fit to the data set has a slope of at least about 5% per milliliter of the fluid. In some embodiments, the slope is from about 6% per milliliter of the fluid to about 9% per milliliter of the fluid. As depicted by the line 622, the best linear fit to the data set has a slope of about 7.5% per milliliter of the fluid. Further, in some embodiments, the best linear fit has a coefficient of determination of at least about 0.8. As depicted by the line 622, the best linear fit has a coefficient of determination of about 0.86.

Referring to FIGS. 5B and 6C, in some embodiments, the first electrode 212 and the second electrode 214 provide a data set (shown by squares in FIG. 6C) correlating the percentage change of the at least one electrical parameter to the extravasated volume of the fluid.

In the graph 630, the at least one electrical parameter is the capacitance. Specifically, in some embodiments, the first electrode 212 and the second electrode 214 provide the data set correlating a percentage change of the capacitance to the extravasated volume of the fluid.

Further, in the graph 630, a best linear fit to the data set is depicted by a line 632. As depicted by the line 632, the best linear fit to the data set has a slope of about 9% per milliliter of the fluid. Further, as depicted by the line 632, the best linear fit has a coefficient of determination of about 0.81.

The first and second electrodes 212, 214 provide a greater magnitude of slope (greater than about 5% per milliliter of the fluid) and a greater coefficient of determination (greater than 0.8), as compared to the conventional sensor patch 100 (shown in FIG. 1A). Thus, the articles 200, 300, 400, 520 (shown in FIGS. 2-5A) including the first and second electrodes 212, 214 may provide improved sensitivity to changes in the at least one electrical parameter. Therefore, the articles 200, 300, 400, 520 may improve detection of extravasation. Further, the improved sensitivity to the changes in the at least one electrical parameter may allow the articles 200, 300, 400, 520 and the systems 500, 550 to reliably detect small changes in the at least one electrical parameter due to small volumes of the fluid extravasated into the tissue.

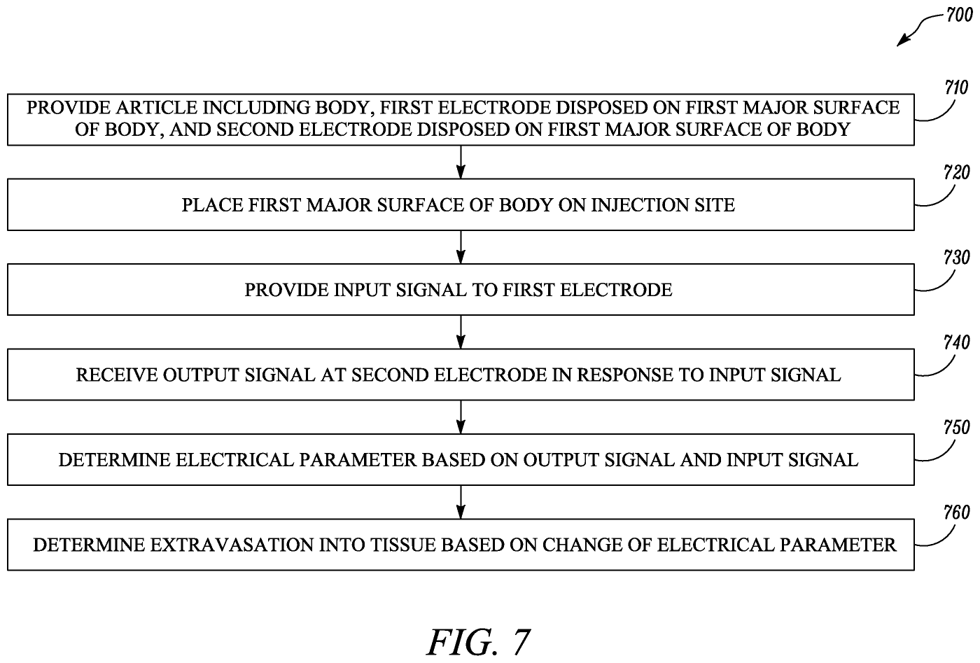

FIG. 7 illustrates a method 700 for detecting extravasation according to an embodiment of the present disclosure. The method 700 may be performed by the systems 500, 550 (shown in FIGS. 5A and 5B, respectively) of the present disclosure. Specifically, in some embodiments, steps involved in the method 700 may be executed by a processing resource, for example, the controller 502 (shown in FIG. 5B) based on instructions stored in a non-transitory computer-readable medium, for example, the memory 508 (shown in FIG. 5B). The method 700 will be described with reference to FIGS. 1-6C. The method 700 includes the following steps:

At step 710, the method 700 includes providing an article including a body, a first electrode including at least one skin-penetrating microfeature and disposed on a first major surface of the body, and a second electrode including at least one skin-penetrating microfeature and disposed on the first major surface of the body. The second electrode is spaced apart from the first electrode.

Referring to FIG. 2, for example, at step 710, the method 700 may include providing the article 200 including the body 202, the first electrode 212 including the at least one skin-penetrating microfeature 213 and disposed on the first major surface 204 of the body 202, and the second electrode 214 including the at least one skin-penetrating microfeature 215 and disposed on the first major surface 204 of the body 202. The second electrode 214 is spaced apart from the first electrode 212.

At step 720, the method 700 further includes placing the first major surface of the body on an injection site. The injection site is disposed between the first electrode and the second electrode.

Referring to FIGS. 3A and 3B, for example, at step 720, the method 700 may further include placing the first major surface 204 of the body 202 on the injection site 360. The injection site 360 is disposed between the first electrode 212 and the second electrode 214.

At step 730, the method 700 further includes providing an input signal across the first electrode and the second electrode. Referring to FIG. 5B, for example, at step 730, the method 700 may further include providing the input signal 504 across the first electrode 212 and the second electrode 214. In some embodiments, the input signal 504 has the frequency ranging from about 0 hertz to about 1 gigahertz.

At step 740, the method 700 further includes determining an output signal across the first electrode and the second electrode in response to the input signal. For example, at step 740, the method 700 may further include determining the output signal 506 across the first electrode 212 and the second electrode 214 in response to the input signal 504.

At step 750, the method 700 further includes determining at least one electrical parameter based on the output signal and the input signal. For example, at step 750, the method 700 further includes determining the at least one electrical parameter based on the output signal 506 and the input signal 504. In some embodiments, the at least one electrical parameter includes one or more of the impedance magnitude, the impedance phase angle, the capacitance, the resistance, and the reactance. In one example, amplitude of the input signal 504 (e.g., |v(t)| of a voltage sinusoid) and the output signal 506 (e.g., |i(t)| of a current sinusoid) can be used to calculate the impedance magnitude (|Z|=|v|/|i|). In another example, a phase difference between the input signal 504 (e.g., |v(t)| of the voltage sinusoid) and the output signal 506 (e.g., |i(t)| of the current sinusoid) can be used to calculate the impedance phase angle (ϕ=angle(v) −angle(i)). Once the impedance magnitude (|Z|) and impedance phase angle (ϕ) are determined, the reactance (X) and the resistance (R) can be calculated from the equation: Z=R+jX, where j is an imaginary number and Z is a complex number including the impedance magnitude (|Z|) and the impedance phase angle (ϕ).

In some embodiments, the method 700 further includes determining the impedance magnitude for a frequency of the input signal 504 ranging from about 1 kilohertz to about 10 kilohertz. In some embodiments, the method 700 further includes determining the impedance magnitude for a frequency of the input signal 504 ranging from about 400 kilohertz to about 1 megahertz. In some embodiments, the method 700 may further include determining the impedance magnitude for a frequency of the input signal 504 of about 1 megahertz.

In some embodiments, the method 700 further includes determining the impedance phase angle for a frequency of the input signal 504 ranging from about 20 kilohertz to about 50 kilohertz. In some embodiments, the method 700 further includes determining the impedance phase angle for a frequency of the input signal 504 ranging from about 200 kilohertz to about 1 megahertz. In some embodiments, the method 700 further includes determining the impedance phase angle for a frequency of the input signal 504 of about 30 kilohertz.

In some embodiments, the method 700 further includes determining the reactance for a frequency of the input signal 504 of about 1 megahertz.

At step 760, the method 700 further includes determining extravasation into the tissue based on a change of the at least one electrical parameter. Referring to FIGS. 3A and 3B, for example, at step 760, the method 700 may further include determining extravasation into the tissue 354 based on the change of the at least one electrical parameter.

In some embodiments, the method 700 further includes generating an alert upon detecting extravasation. Referring to FIG. 5B, for example, the alert may be generated by the controller 502.

In some embodiments, the method 700 further includes providing a securement device on a second major surface of the body. In some embodiments, the method 700 further includes detachably coupling an intravenous catheter with the securement device. Referring to FIG. 4, for example, the method 700 may further include providing the securement device 450 on the second major surface 206 of the body 202. The method 700 may further include detachably coupling the intravenous catheter 470 with the securement device 450.

In some embodiments, the method 700 further includes determining at least one of the extravasated volume of the fluid and the time period of extravasation based on the change in the at least one electrical parameter.

Referring to FIGS. 5B and 6A, in some embodiments, the method 700 further includes determining, for the input signal 504 having a frequency of about 1 megahertz, the data set (shown by squares in FIG. 6A) correlating the percentage change of the at least one electrical parameter to the extravasated volume of the fluid. In some embodiments, the best linear fit (shown by line 612 in FIG. 6A) to the data set has the slope of at least about 5% per milliliter of the fluid. In some embodiments, the slope is from about 6% per milliliter of the fluid to about 9% per milliliter of the fluid. In some embodiments, the best linear fit has a coefficient of determination of at least about 0.8.

### Experimental Results

Experiments were performed on a pig to determine a performance of electrodes in determining extravasation. Specifically, the experiments were carried out on four injection sites in a femoral vein area of the pig.

In a first experiment, a pair of wet electrodes was placed at each of the four injection sites, such that each pair of wet electrodes flanked the femoral vein at a corresponding injection site of the four injection sites. A pair of Red Dot 2360 electrodes (available from the 3M company) were used as the pair of wet electrodes in the first experiment. A catheter was inserted into the IV site and positioned such that when injected, saline would be deposited extra-venously, mimicking extravasation.

Prior to injection of saline, an input signal (a constant-amplitude sinusoidal voltage) was excited across the two wet electrodes. A frequency of the input signal was varied from about 100 hertz to about 1 megahertz. Further, an output signal was determined (i.e., the resulting sinusoidal current through the two wet electrodes). Based on the input signal and the output signal, an impedance magnitude and an impedance phase angle were recorded at each injection site. The impedance magnitude and the impedance phase angle recorded at each injection site prior to injection of saline may be referred to as a baseline impedance magnitude and a baseline impedance phase angle, respectively.

Subsequently, two milliliters of saline was extra-venously injected into a corresponding tissue at each injection site. Shifts in the impedance magnitude from the baseline impedance magnitude and the impedance phase angle from the baseline impedance phase angle were observed.

Specific frequencies of the input signal were identified for which shifts in an average impedance magnitude at the four injection sites due to the saline injections were greatest and most statistically different (i.e., most distinguishable from the baseline impedance magnitude). It was determined that the shifts in the average impedance magnitude at the four injection sites were greatest at a frequency of the input signal ranging from about 10 kilohertz to about 100 kilohertz.

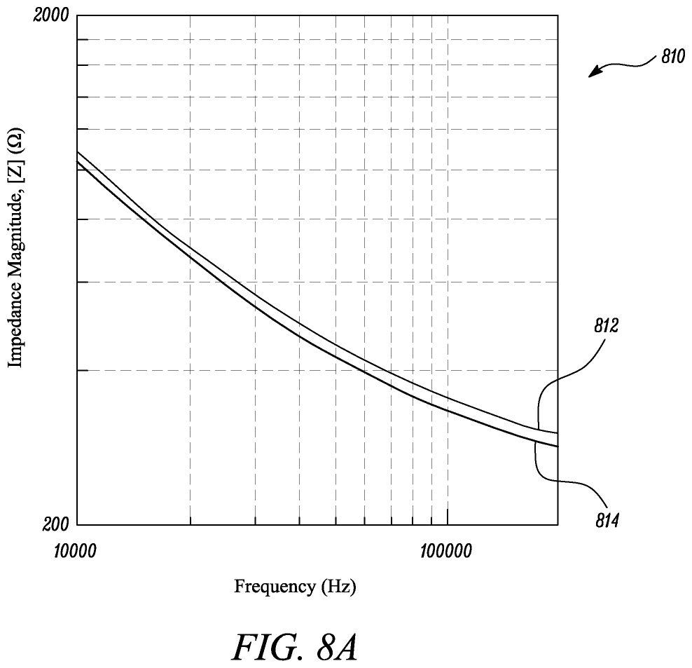

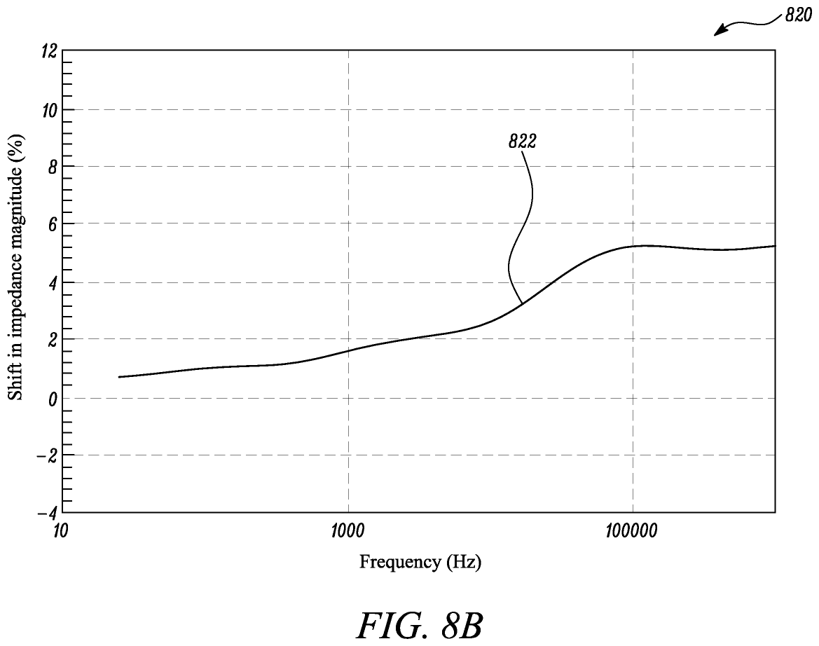

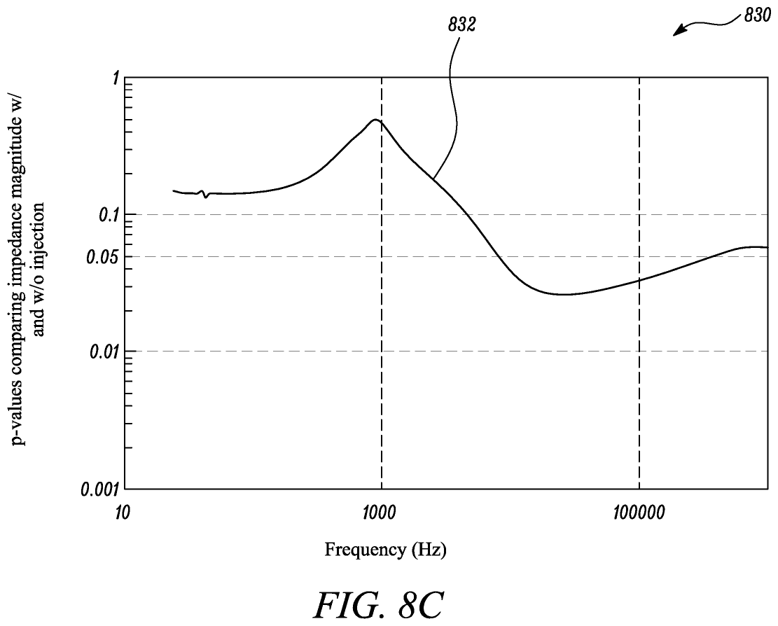

FIG. 8A illustrates a graph 810 depicting a variation of impedance magnitude with respect to frequency measured using wet electrodes. Specifically, the graph 810 depicts the variation of the average impedance magnitude at the four injection sites with respect to a frequency of the input signal ranging from about 10 kilohertz to about 100 kilohertz.

The graph 810 includes a first curve 812 corresponding to the average impedance magnitude at the four injection sites when no saline was injected. The graph 810 further includes a second curve 814 corresponding to the average impedance magnitude at the four injection sites after two milliliters of saline was injected. A percentage shift of the average impedance magnitude between the first curve 812 and the second curve 814 was determined.

FIG. 8B illustrates a graph 820 depicting a variation of the percentage shift in impedance magnitude with respect to frequency. The graph 820 includes a curve 822 corresponding to the percent shift of the average impedance magnitude between the first curve 812 and the second curve 814 (shown in FIG. 8A). As depicted by the curve 822, it was determined that a percentage shift between the first curve 812 and the second curve 814 was about 5.3% for the input signal having a frequency of about 100 kilohertz.

FIG. 8C illustrates a graph 830 depicting a variation of p-values with respect to frequency. The graph 830 includes a curve 832 corresponding to p-values representing a degree of statistical difference between the first curve 812 and the second curve 814 (shown in FIG. 8A). The p-values may be minimized at most prominent and statistically significant impedance magnitude shifts. As depicted by the curve 832, it was determined that the p-value was less than about 0.05 for the input signal having a frequency of about 100 kilohertz.

Therefore, the input signal having a frequency of about 100 kilohertz provided statistically significant impedance magnitude shifts, which may be monitored to detect extravasation.

Further, specific frequencies of the input signal were identified for which shifts in the average impedance phase angle due to the saline injections were greatest and most statistically different (i.e., most distinguishable from the baseline impedance phase angle). It was determined that the shifts in the average impedance phase angle at the four injection sites were greatest at a frequency of the input signal ranging from about 100 hertz to about 1 kilohertz, and a frequency of the input signal ranging from about 40 kilohertz to about 70 kilohertz.

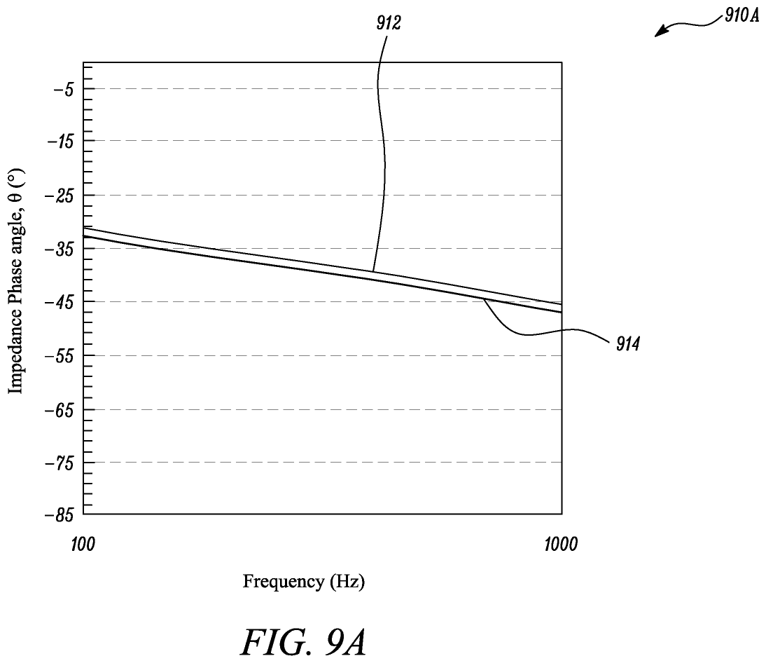

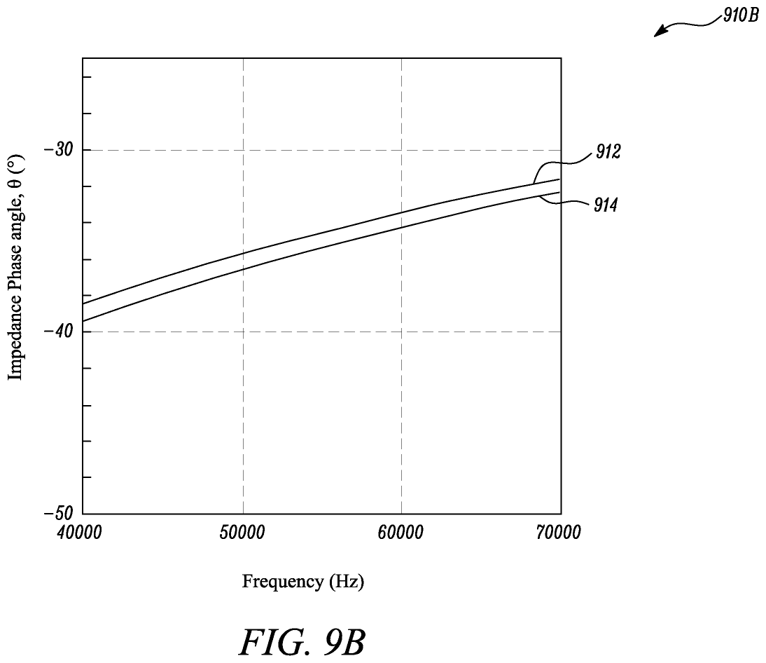

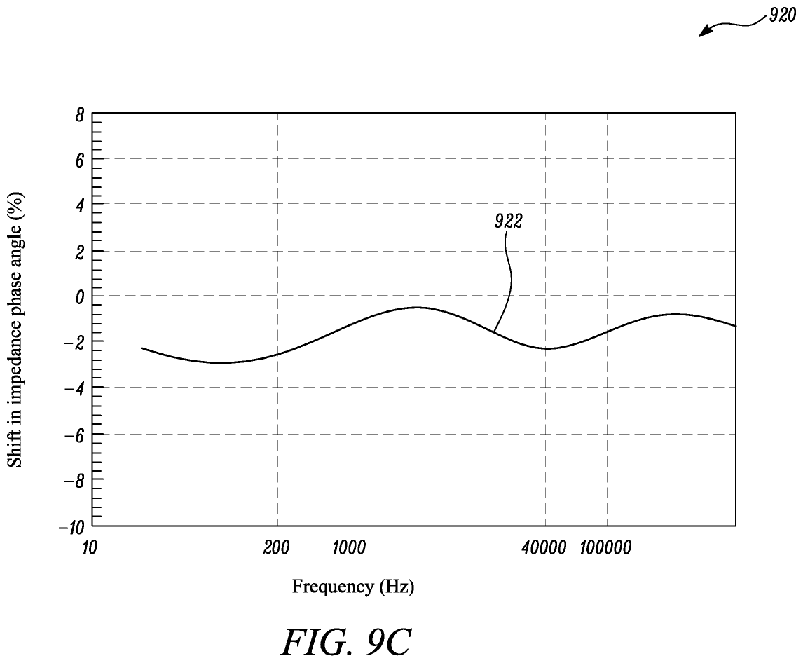

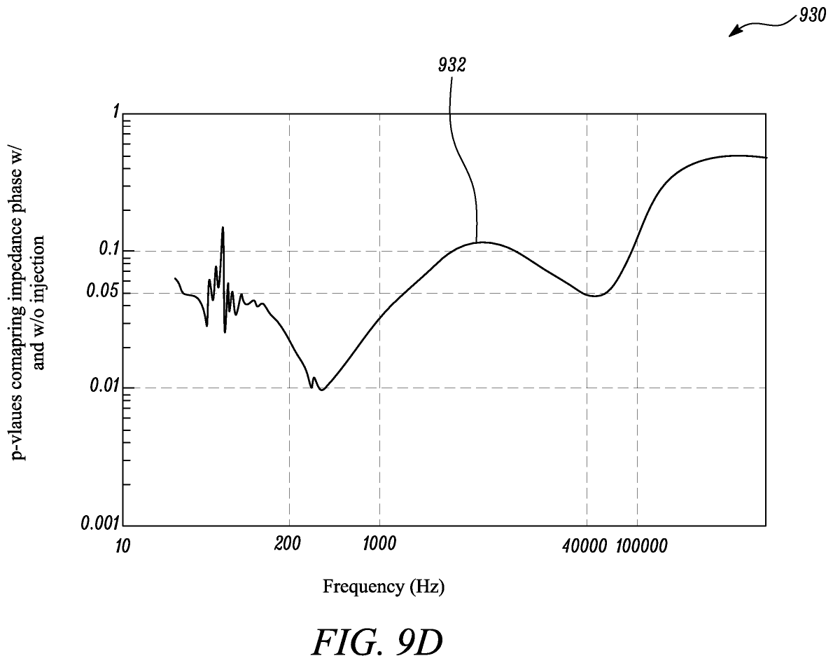

FIGS. 9A and 9B illustrate graphs 910A, 910B, respectively, depicting a variation of impedance phase angle with respect to frequency. Specifically, the graph 910A depicts the variation of the average impedance phase angle at the four injection sites with respect to a frequency of the input signal ranging from about 100 hertz to about 1 kilohertz. Further, the graph 920B depicts the variation of the average impedance phase angle at the four injection sites with respect to a frequency of the input signal ranging from about 40 kilohertz to about 70 kilohertz.

The graphs 910A, 910B include a first curve 912 corresponding to the average impedance phase angle at the four injection sites when no saline was injected. The graphs 910A, 910B further include a second curve 914 corresponding to the average impedance phase angle at the four injection sites after two milliliters of saline was injected. A percentage shift of the average impedance phase angle between the first curve 912 and the second curve 914 was determined.

FIG. 9C illustrates a graph 920 depicting a variation of the percentage shift in impedance phase angle with respect to frequency. The graph 920 includes a curve 922 corresponding to the percentage shift of the average impedance phase angle between the first curve 912 and the second curve 914 (shown in FIGS. 9A and 9B). As depicted by the curve 922, it was determined that a first percentage shift between the first curve 912 and the second curve 914 was about −2.7% for the input signal having a frequency of about 200 hertz, and a second percentage shift between the first curve 912 and the second curve 914 was about −2.2% for the input signal having a frequency of about 40 kilohertz.

FIG. 9D illustrates a graph 930 depicting a variation of p-values with respect to frequency. The graph 930 includes a curve 932 corresponding to p-values representing a degree of statistical difference between the first curve 912 and the second curve 914 (shown in FIGS. 9A and 9B). The p-values may be minimized at most prominent and statistically significant impedance phase angle shifts. As depicted by the curve 932, it was determined that the p-value was less than about 0.05 the input signal having a frequency of about 200 hertz and about 40 kilohertz.

It was noted that a maximum percentage shift in the average impedance phase angle due to 2 milliliter saline was less than a maximum percentage shift in the average impedance magnitude (−2.7% as compared to 5.3%). It was therefore concluded that the wet electrodes had a lower sensitivity to shifts in the impedance phase angle at the four injection sites as compared to shifts in the impedance magnitude.

In a second experiment, two milliliters of saline was extra-venously injected into the tissue at each injection site in increments of one milliliter. A pair of Red Dot 2360 electrodes were used as the pair of wet electrodes in the second experiment. For the second experiment, a frequency of the input signal was set at 100 kilohertz (as it provided most statistically significant impedance magnitude shifts). The impedance magnitude at each injection site was recorded after one milliliter of saline was injected, and after two milliliters of saline was injected. Further, a percentage shift was calculated based on shifts in the impedance magnitude at each injection site from the baseline impedance magnitude. The impedance magnitude recorded at each injection site and the percentage shift calculated are provided in Table 1 below.

TABLE 1

| | Sites | | | | | | | |
|---|---|---|---|---|---|---|---|---|
| | Site 1 | | Site 2 | | Site 3 | | Site 4 | |
| | \|Z\| | Δ\|Z\| | \|Z\| | Δ\|Z\| | \|Z\| | Δ\|Z\| | \|Z\| | Δ\|Z\| |
| Parameter | (Ω) | (%) | (Ω) | (%) | (Ω) | (%) | (Ω) | (%) |
| 0 ml | 315.2 | — | 345 | — | 380 | — | 370 | — |
| 1 ml | 312.4 | 0.89 | 339 | 1.74 | 364 | 4.21 | 355 | 4.05 |
| 2 ml | 311.3 | 1.23 | 332.5 | 3.62 | 347.5 | 8.55 | 341 | 7.83 |

Referring to Table 1, impedance magnitude values at 100 kilohertz reduced monotonically as more saline was extra-venously injected consistently across all four injection sites. Impedance magnitude shifts at 100 kilohertz resulting from 2 mL saline injection over bassline impedance magnitude were as low as 1.23% (Site 1, 2 mL) and as high as 8.55% (Site 3, 2 mL). Average percentage shift in impedance magnitude at the four injection sites with respect to a volume of injected saline was then calculated.

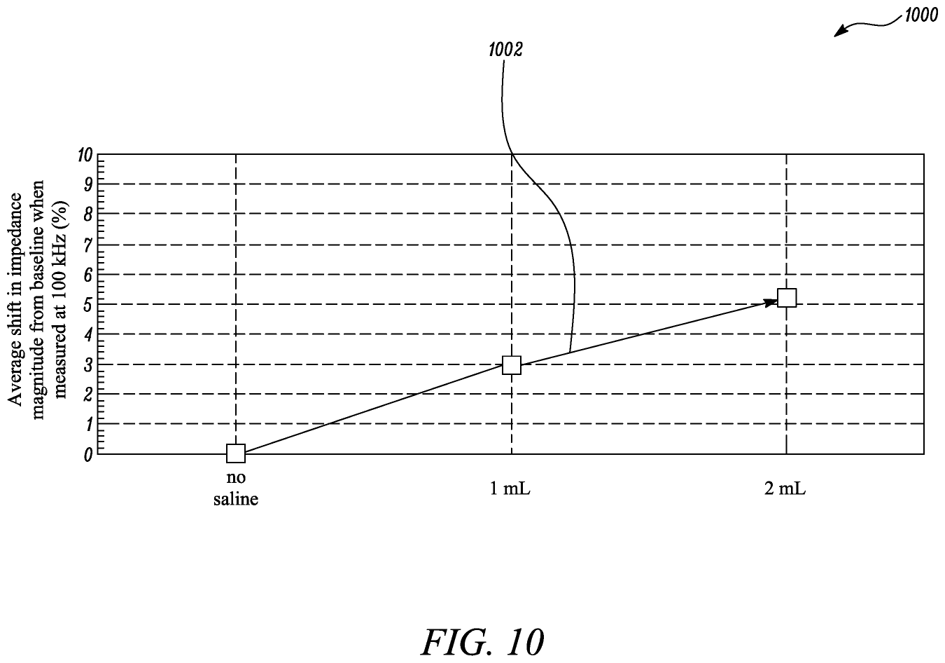

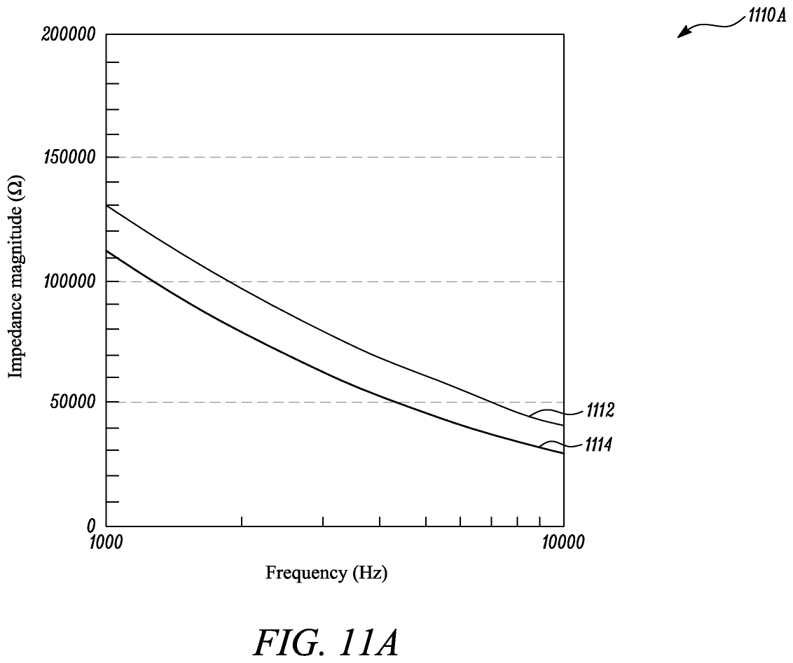

FIG. 10 illustrates a graph 1000 depicting a variation of an average percentage shift in impedance magnitude at the four injection sites with respect to volume of injected saline.

The graph 1000 includes a curve 1002 depicting the average percentage shift in impedance magnitude of the four injection sites with respect to volume of injected saline derived from Table 1. As depicted by the curve 1002, the average percentage shift in impedance magnitude of the four injection sites when 1 milliliter of saline was injected was about 3%. Further, as depicted by the curve 1002, the average percentage shift in impedance magnitude of the four injection sites when 2 milliliter of saline was injected was about 5.2%.

It was noted that the average percentage shift in the impedance magnitude at the four injection sites was low. It was therefore concluded that the wet electrodes could not be used to reliably differentiate between 1 milliliter of saline and 2 milliliters of saline.

In a third experiment, a pair of dry electrodes was placed at each of the four injection sites, such that the pair of dry electrodes flanked the femoral vein at a corresponding injection site of the four injection sites. The first and second electrodes 212, 214 of the present disclosure were used as the pair of dry electrodes in the third experiment.

Prior to injection of saline, an input signal (a constant-amplitude voltage sinusoid) was provided across the dry electrodes. A frequency of the input signal was varied from about 100 hertz to about 1 megahertz. Further, an output signal was determined (i.e., the sinusoidal current running through the pair of dry electrodes). Based on the input signal and the output signal, an impedance magnitude and an impedance phase angle were recorded at each injection site. The impedance magnitude and the impedance phase angle at each injection site prior to injection of saline may be referred to as a baseline impedance magnitude and a baseline impedance phase angle, respectively.

Subsequently, two milliliters of saline was extra-venously injected into the corresponding tissue at each injection site. Shifts in the impedance magnitude from the baseline impedance magnitude and the impedance phase angle from the baseline impedance phase angle were observed.

Specific frequencies of the input signal were identified for which shifts in an average impedance magnitude due to the saline injections were greatest and most statistically different (i.e., most distinguishable from the baseline impedance magnitude). It was determined that the shifts in the average impedance magnitude at the four injection sites were the greatest at a frequency of the input signal ranging from about 1 kilohertz to about 10 kilohertz, and a frequency of the input signal ranging from about 400 kilohertz to about 1 megahertz.

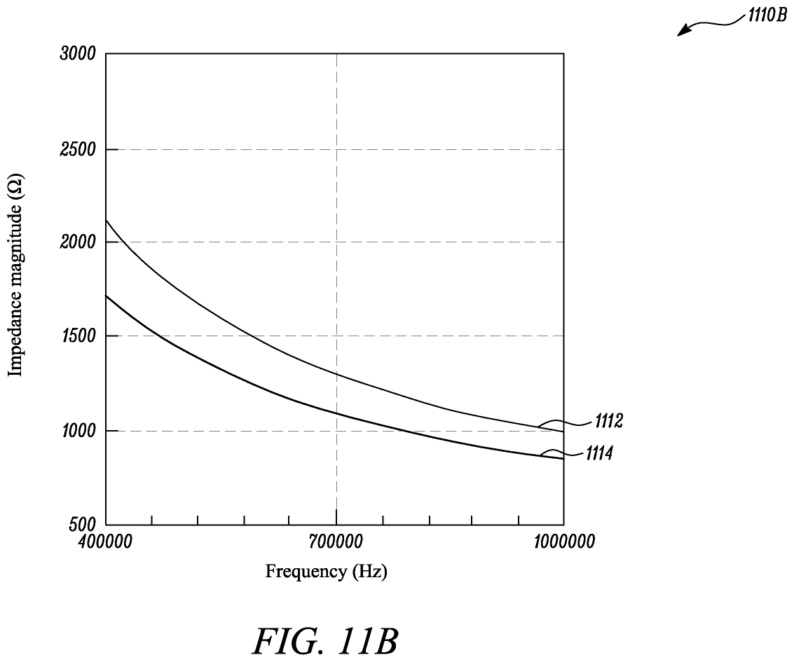

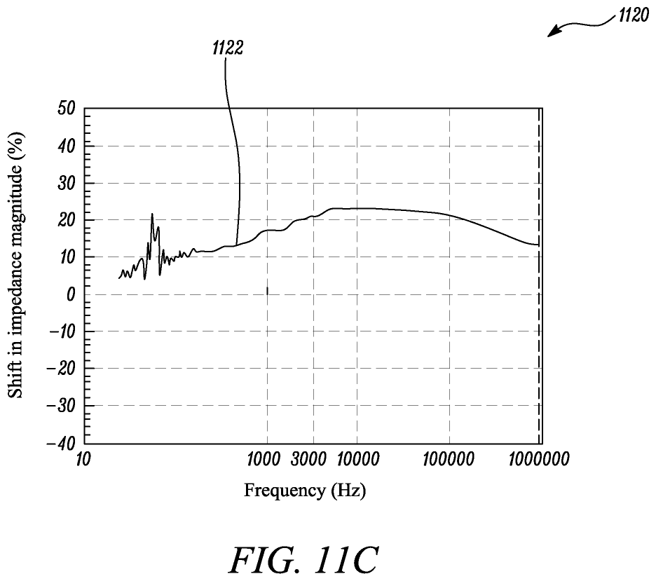

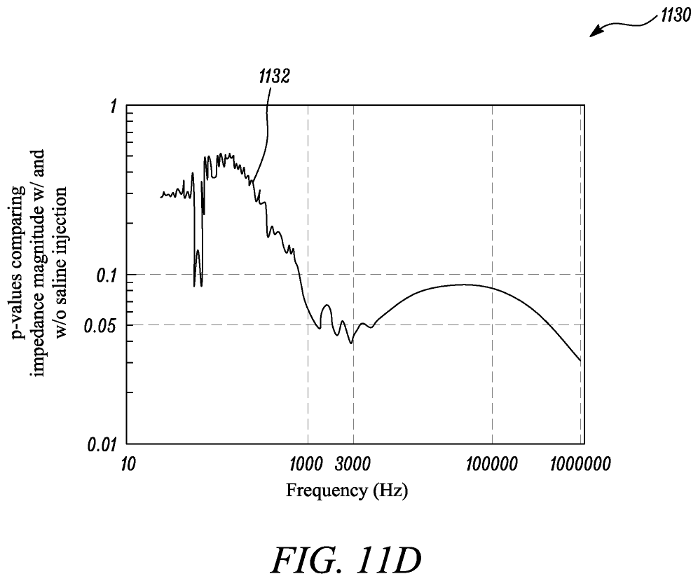

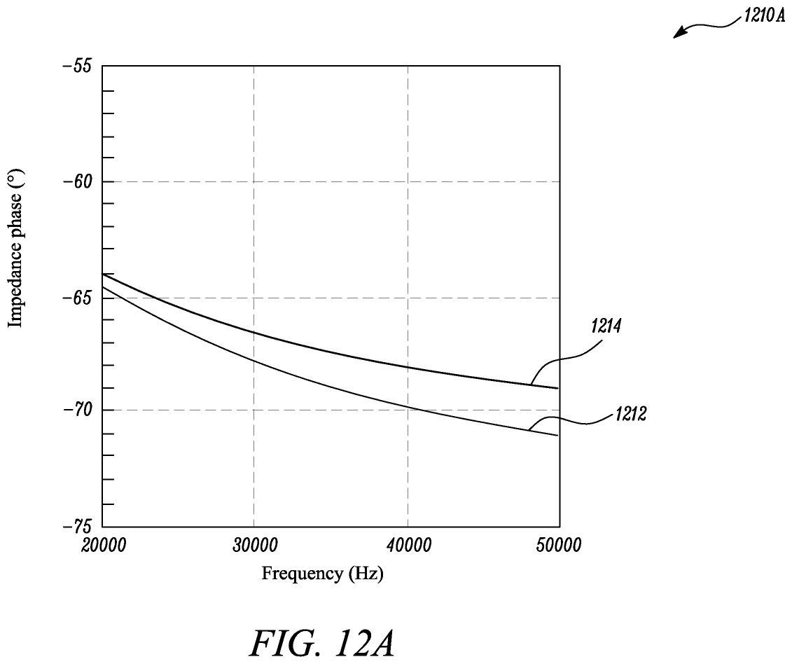

FIGS. 11A and 11B illustrate graphs 1110A, 1110B depicting a variation of impedance magnitude with respect to frequency measured using dry electrodes. Specifically, the graph 1110A depicts the variation of the average impedance magnitude at the four injection sites with respect to a frequency of the input signal ranging from about 1 kilohertz to about 10 kilohertz, and the graph 1110B depicts the average impedance magnitude at the four injection sites with respect to a frequency of the input signal ranging from about 400 kilohertz to about 1 megahertz.

The graphs 1110A, 1110B include a first curve 1112 corresponding to the average impedance magnitude recorded at the four injection sites when no saline was injected. The graphs 1110A, 1110B further include a second curve 1114 corresponding to the average impedance magnitude at the four injection sites after two milliliters of saline was injected. A percentage shift of the average impedance magnitude between the first curve 1112 and the second curve 1114 was determined.

FIG. 11C illustrates a graph 1120 depicting a variation of percentage shift in impedance magnitude with respect to frequency. The graph 1120 includes a curve 1122 corresponding to the percentage shift of the average impedance magnitude between the first curve 1112 and the second curve 1114 (shown in FIGS. 11A and 11B). As depicted by the curve 1122, it was determined that a first percentage shift between the first curve 1112 and the second curve 1114 was about 20.9% for the input signal having a frequency of about 3 kilohertz, a second percentage shift between the first curve 1112 and the second curve 1114 was about 23.5% for the input signal having a frequency of about 10 kilohertz, and a third percentage shift between the first curve 1112 and the second curve 1114 was about 13.6% at 1 megahertz.

FIG. 11D illustrates a graph 1130 depicting a variation of p-values with respect to frequency. The graph 1130 includes a curve 1132 corresponding to p-values representing a degree of statistical difference between the first curve 1112 and the second curve 1114 (shown in FIGS. 11A and 11B). The p-values may be minimized at most prominent and statistically significant impedance magnitude shifts. As depicted by the curve 1132, it was determined that the p-value was less than about 0.05 for the input signal having a frequency of about 3 kilohertz and about 1 megahertz.

Therefore, the input signal having a frequency of about 1 megahertz provided statistically significant impedance magnitude shifts at the four injection sites, which may be monitored to detect extravasation, and determine an extravasated volume of the saline.

Moreover, specific frequencies of the input signal were identified for which shifts in the average impedance phase angle at the four injection sites due to the saline injections were greatest and most statistically different (i.e., most distinguishable from the baseline impedance phase angle). It was determined that the shifts in the average impedance phase angle at the four injection sites were greatest at frequency of the input signal ranging from about 20 kilohertz to about 50 kilohertz, and a frequency of the input signal ranging from about 200 kilohertz to about 1 megahertz.

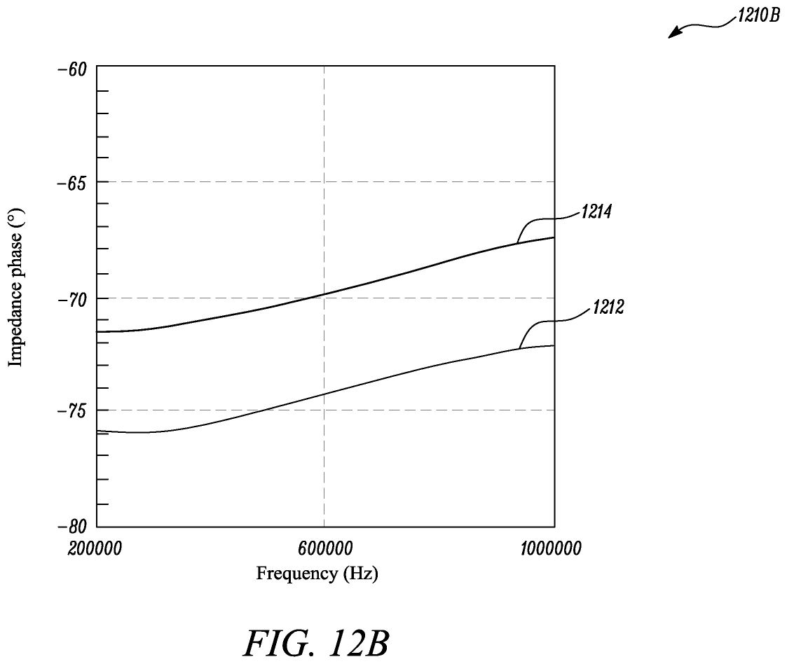

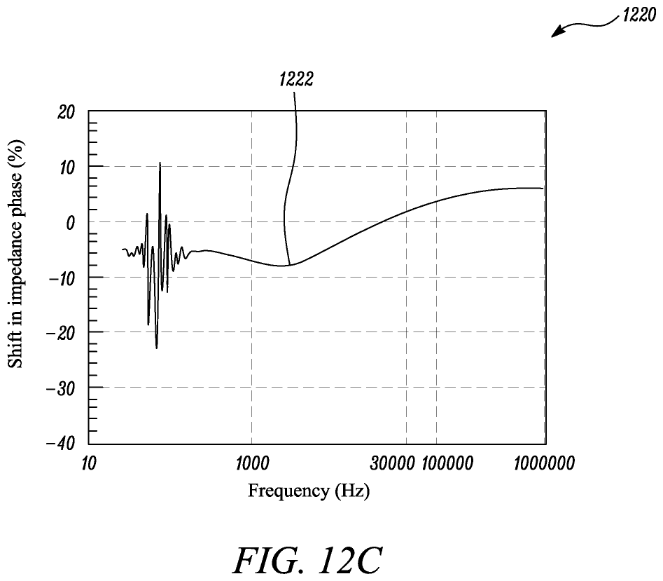

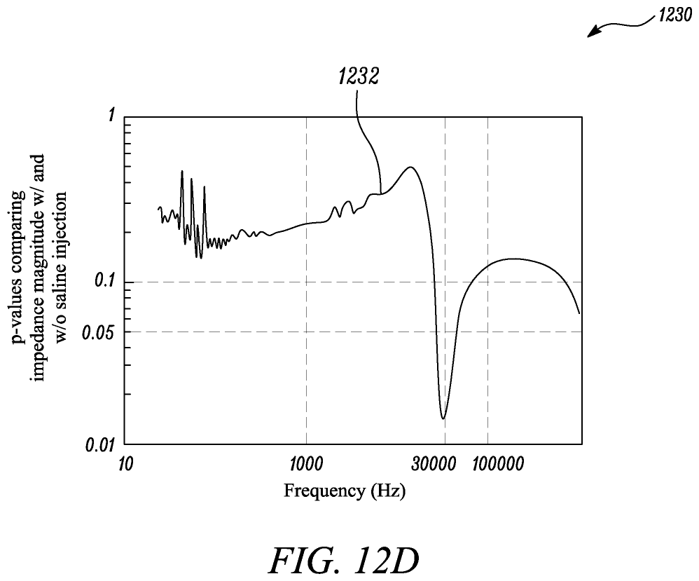

FIGS. 12A and 12B illustrate graphs 1210A, 1210B depicting a variation of impedance phase angle with respect to frequency measured using dry electrodes. Specifically, the graph 1210A depicts the average phase angle at the four injection sites with respect to a frequency of the input signal ranging from about 20 kilohertz to about 50 kilohertz, and the graph 1210B depicts the average impedance phase angle at the four injection sites with respect to a frequency of the input signal ranging from about 200 kilohertz to about 1 megahertz.

The graphs 1210A, 1210B include a first curve 1212 corresponding to the average impedance magnitude recorded at the four injection sites when no saline was injected. The graphs 1210A, 1210B further include a second curve 1214 corresponding to the average impedance magnitude at the four injection sites after two milliliters of saline was injected. A percentage shift of the average impedance phase angle between the first curve 1212 and the second curve 1214 was determined.

FIG. 12C illustrates a graph 1220 depicting a variation of percentage shift in impedance phase angle with respect to frequency. The graph 1220 includes a curve 1222 corresponding to the percentage shift of the average impedance phase angle between the first curve 1212 and the second curve 1214 (shown in FIGS. 12A and 12B). As depicted by the curve 1222, it was determined that a first percentage shift between the first curve 1212 and the second curve 1214 was about 2.5% for the input signal having a frequency of about 30 kilohertz, and a second percentage shift between the first curve 1212 and the second curve 1214 was about 6.4% for the input signal having a frequency of about 1 megahertz.

FIG. 12D illustrates a graph 1230 depicting a variation of p-values with respect to frequency. The graph 1230 includes a curve 1232 corresponding to p-values representing a degree of statistical difference between the first curve 1212 and the second curve 1214 (shown in FIGS. 12A and 12B). The p-values may be minimized at most prominent and statistically significant impedance phase angle shifts. As depicted by the curve 1232, it was determined that the p-value was less than about 0.05 for the input signal having a frequency of about 30 kilohertz.

Therefore, the input signal having a frequency of about 30 kilohertz provided statistically significant impedance phase angle shifts, which may be monitored to detect extravasation and determine an extravasated volume of the saline using dry electrodes.

In a fourth experiment, two milliliters of saline was extra-venously injected into the tissue at each injection site in increments of one milliliter. The first and second electrodes 212, 214 of the present disclosure were used as the pair of dry electrodes in the fourth experiment. For the fourth experiment, a frequency of the input signal was set at 1 megahertz (as it provided most statistically significant impedance magnitude shifts). The impedance magnitude at each injection site was recorded after one milliliter of saline was injected, and after two milliliters of saline was injected. Further, a percentage shift was calculated based on shifts in the impedance magnitude at each injection site from the baseline impedance magnitude. The impedance magnitude recorded at each injection site and the percentage shift calculated are provided in Table 2 below.

TABLE 2

| | Sites | | | | | | | |
|---|---|---|---|---|---|---|---|---|
| | Site 1 | | Site 2 | | Site 3 | | Site 4 | |
| | \|Z\| | Δ\|Z\| | \|Z\| | Δ\|Z\| | \|Z\| | Δ\|Z\| | \|Z\| | Δ\|Z\| |
| Parameter | (Ω) | (%) | (Ω) | (%) | (Ω) | (%) | (Ω) | (%) |
| 0 ml | 593 | — | 1305 | — | 1350 | — | 680 | — |
| 1 ml | 567 | 4.38 | 1240 | 4.98 | 1315 | 2.59 | 646 | 5.00 |
| 2 ml | 520 | 12.31 | 1045 | 19.92 | 1165 | 13.70 | 615 | 9.55 |

Referring to Table 2, impedance magnitude values at 1 megahertz reduced monotonically as more saline was extra-venously injected consistently across all four injection sites. These changes in in impedance magnitude represent a substantial shift (about 20%) from the baseline impedance magnitude (Site 3, at 2 ml). Average percentage shift in impedance magnitude at the four injection sites with respect to a volume of injected saline was then calculated.

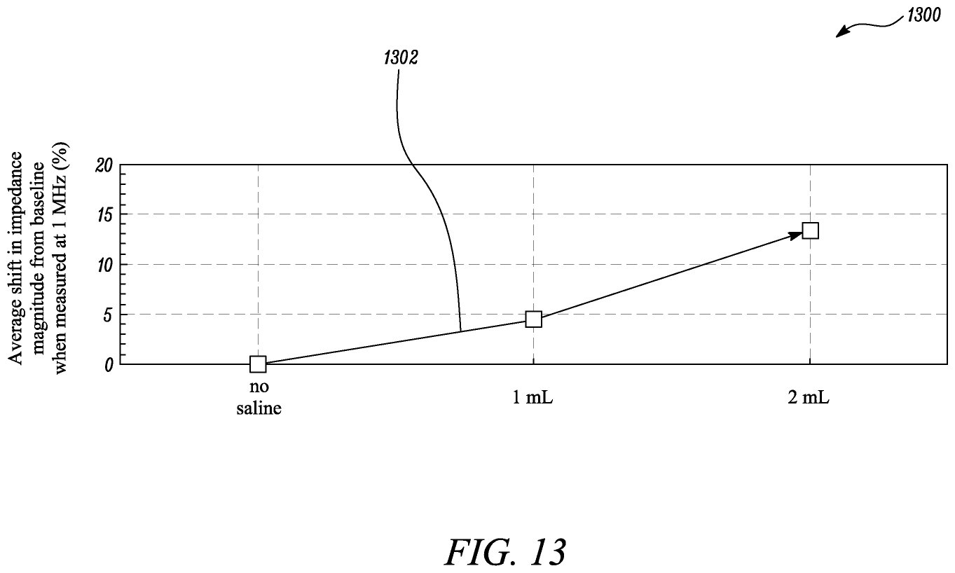

FIG. 13 illustrates a graph 1300 depicting a variation of a percentage shift in impedance magnitude with respect to volume.

The graph 1300 includes a curve 1302 depicting an average percentage shift in impedance magnitude with respect to a volume of injected saline derived from Table 2 provided above. As depicted by the curve 1302, the average percentage shift in impedance magnitude of the four injection sites when 1 milliliter of saline was injected was about 4.5%. Further, as depicted by the curve 1302, the average percentage shift in impedance magnitude of the four injection sites when 2 milliliters of saline was injected was about 13.4%.

Notably, these shifts were highly statistically differentiable from one another, with shifts resulting from 1 mL injections posting p=0.0016, shifts resulting from an additional 1 mL injection (i.e., difference between 1 mL and 2 mL extra-venous injection) posting p=0.011. This combination of consistent monotonicity and strong statistical differentiability between different volumes of extra-venous injections provided a promising method for quantifying the volume of infiltrate during monitoring, which might be a valuable clinical metric.

It was also noted that the average percentage shift in the impedance magnitude at the four injection sites was much higher than wet electrodes. It was therefore concluded that the dry electrodes could be used to reliably differentiate between 1 milliliter of saline and 2 milliliters of saline.

As is apparent from Table 2, shifts in impedance magnitude at each injection site increased monotonically with increasing amounts of extra-venously injected saline at a frequency of the input signal of about 1 megahertz. It was further noticed that impedance phase angle also increased monotonically with increasing injection volumes. It was therefore hypothesized that examining complex impedance (i.e., both real (resistive) and imaginary (reactive) components of impedance) might provide a particularly strong and statistically significant shifts upon extravasation.

In a fifth experiment, a resistance and a reactance at the four injection sites were determined using dry electrodes. The first and second electrodes 212, 214 of the present disclosure were used as the pair of dry electrodes in the fifth experiment. For the fifth experiment, a frequency of the input signal was set at 1 megahertz. The resistance and the reactance at each injection site were recorded after one milliliter of saline was injected, and after two milliliters of saline was injected. Further, a percentage shift was calculated based on shifts in the reactance at each injection site. The resistance and reactance recorded, and the percentage shift calculated at each injection site is provided in Table 3 below.

TABLE 3

| | Site | | | | | | | | | | | |
|---|---|---|---|---|---|---|---|---|---|---|---|---|
| | Site 1 | | | Site 2 | | | Site 3 | | | Site 4 | | |
| | R | jX | ΔjX | R | jX | ΔjX | R | jX | ΔjX | R | jX | ΔjX |
| Par. | (Ω) | (Ω) | (%) | (Ω) | (Ω) | (%) | (Ω) | (Ω) | (%) | (Ω) | (Ω) | (%) |
| 0 ml | 166 | −568 | — | 440 | −1230 | — | 410 | −1290 | — | 207.2 | −644 | — |
| 1 ml | 188 | −536 | 5.63 | 415 | −1170 | 4.88 | 425 | −1240 | 3.87 | 205 | −612 | 4.96 |
| 2 ml | 188 | −486 | 14.43 | 540 | −912 | 25.85 | 377 | −1120 | 13.18 | 207.6 | −581 | 9.9 |

Referring to Table 3, at 1 megahertz, reactance values consistently moved towards less negative values of electrical reactance for the four injection sites. Shifts of the reactance at the four injection sites were strong (between 5% and 26%) and increased monotonically with increasing amounts of extra-venous saline injection.

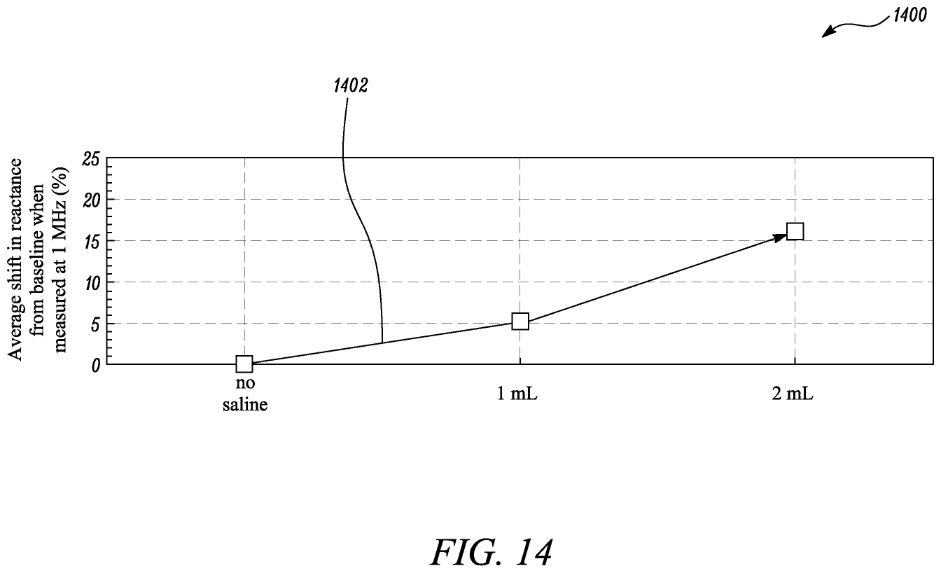

FIG. 14 illustrates a graph 1400 depicting a variation of a percentage shift in reactance with respect to volume.

The graph 1400 includes a curve 1402 depicting an average percentage shift in reactance with respect to a volume of injected saline derived from Table 3 provided above. As depicted by the curve 1402, the average percentage shift in the reactance of the four injection sites when 1 milliliter of saline was injected was about 5.2%. Further, as depicted by the curve 1402, the average percentage shift in impedance magnitude of the four injection sites when 2 milliliters of saline was injected was about 16.2%.

Shifts in the reactance of the tissues at the four injection sites were statistically distinguishable from one another with the following p-values:
No saline vs. 1 mL saline: p=0.00087;
No saline vs. 2 mL saline: p=0.0095; and
1 mL saline vs. 2 mL saline: p=0.024.

Thus, it was concluded that measurement of reactance at the injection sites at 1 megahertz using the dry electrodes could detect extravasation with high sensitivity and can be used to quantify volume of fluid extravasated into tissue vs. time. When using the dry electrodes, reactance at the four injection sites measured at 1 megahertz provided strong, statistically significant, and monotonically increasing shifts when small volumes of saline were extra-venously injected.

The negative phase angles (see graphs 1210A and 1210B of FIGS. 12A and 12B, respectively) and negative reactance values (see Table 3) indicated that the four injection sites exhibited electrically capacitive properties. Shifts in negative reactance values due to injection of different saline volumes corresponded directly to shifts in capacitance as given by the equation below:

$${C}_{IV} = \frac{1}{2 π {fX}_{IV}}$$

Where C_{IV} is the capacitance at an injection site, X_{IV} is the reactance at the injection site, and f is the frequency of the input signal.

In a sixth experiment, the capacitance was calculated at the four injection sites. The first and second electrodes 212, 214 of the present disclosure were used as the pair of dry electrodes in the sixth experiment. For the four injection sites, the relationship provided above provided was used to calculate capacitance at the four injection sites when no saline, 1 mL of saline, and 2 mL of saline were extra-venously injected. Further, a percentage shift was calculated based on shifts in the capacitance at each injection site. The calculated capacitances and the percentage shift calculated at the four injection sites are provided in Table 4 below.

TABLE 4

| | Sites | | | | | | | |
|---|---|---|---|---|---|---|---|---|
| | Site 1 | | Site 2 | | Site 3 | | Site 4 | |
| | C1 | ΔC1 | C2 | ΔC2 | C3 | ΔC3 | C4 | ΔC4 |
| Parameter | (pF) | (%) | (pF) | (%) | (pF) | (%) | (pF)) | (%) |
| 0 ml | 280 | — | 127 | — | 123.5 | — | 246.5 | — |
| 1 ml | 300 | 7.14 | 136 | 7.08 | 128 | −3.64 | 260.4 | −5.64 |
| 2 ml | 330 | 17.85 | 173 | 36.22 | 143.5 | −16.19 | 274 | −11.16 |

Referring to Table 4, the capacitances consistently increased monotonically across all four injection sites and represented relatively strong shifts in percentage value from baseline, reaching values as high as 36% (site 2 in Table 4).

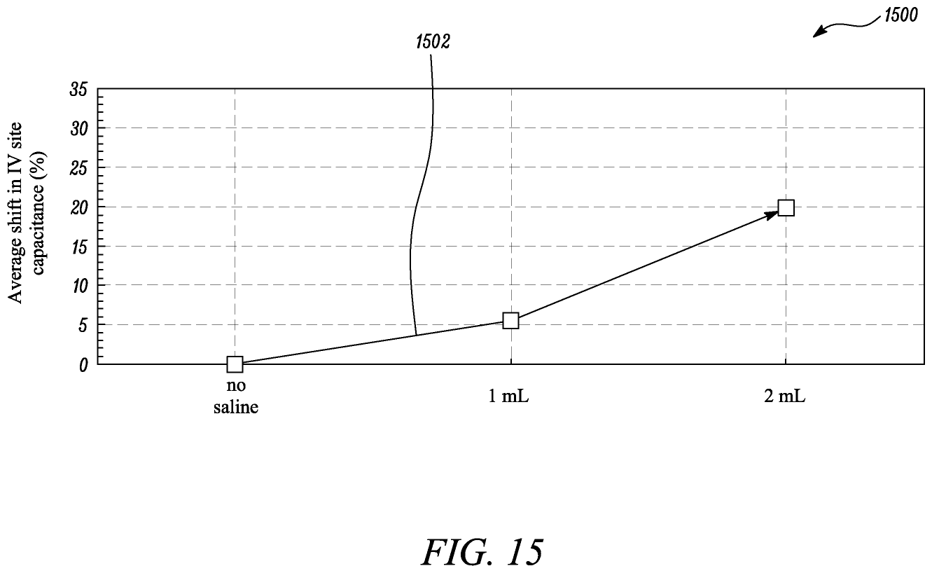

FIG. 15 illustrates a graph 1500 depicting a variation of a percentage shift in capacitance with respect to volume.

The graph 1500 includes a curve 1502 depicting an average percentage shift in capacitance at the four injection sites with respect to a volume of injected saline derived from Table 4 provided above. As depicted by the curve 1502, the average percentage shift in the capacitance of the four injection sites when 1 milliliter of saline was injected was about 5.5%. Further, as depicted by the curve 1302, the average percentage shift in impedance magnitude of the four injection sites when 2 milliliters of saline was injected was about 20%.

Presence of 1 mL extra-venous saline was strikingly distinguishable from the “no saline” condition, with a p-value of 0.0009. Further, 1 mL vs. 2 mL of extra-venous injections were distinguishable with a p-value of 0.035. Thus, it was concluded that measurement of capacitance at the injection sites using the dry electrodes could detect extravasation with high sensitivity and can be used to quantify volume of fluid extravasated into tissue vs. time. When using the dry electrodes, capacitance at the four injection sites provided strong, statistically significant, and monotonically increasing shifts when small volumes of saline were extra-venously injected.

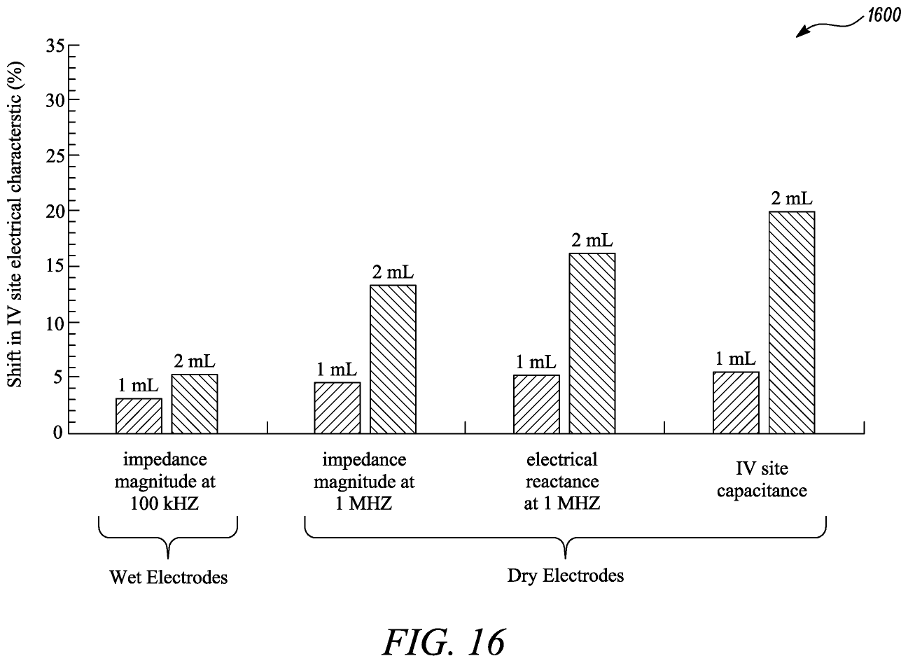

FIG. 16 illustrates a graph 1600 depicting a percentage change of electrical characteristics measured using a pair of wet electrodes and a pair of dry electrodes.

As depicted by the graph 1600, the wet electrodes provided modest shifts in impedance magnitude. Further, as depicted by graph 920 of FIG. 9C, the wet electrodes provided weak shifts in impedance phase angle. The lack of sensitivity provided by the wet electrodes was likely due to the large impedance presented by the skin's top layer (i.e., the stratum corneum) dominating the impedance measurement. This made it difficult to detect small changes in impedance at the four injection sites.

Additionally, because dry electrodes picked up large shifts in both impedance magnitude and phase angle, this enabled even larger shifts to be observed when measuring electrical properties of the four injection sites related to complex impedance (i.e., reactance and capacitance). It was observed that shifts resulting from small volumes of extra-venous saline injection for electrical reactance and capacitance were more pronounced than impedance magnitude-related shifts. These shifts in reactance and capacitance measured using dry electrodes were much more pronounced than the impedance magnitude measured using wet electrodes.

Beyond enhanced sensitivity, the ability to distinguish between 1 mL and 2 mL injections was also enhanced when using dry electrodes. Thus, using dry electrodes to monitor the injection site electrical characteristics provided enhanced sensitivity to infiltrate detection and ability to track infiltrate. The use of dry electrodes therefore provided enhanced sensitivity to the detection of extra-venously injected saline.

Unless otherwise indicated, all numbers expressing feature sizes, amounts, and physical properties used in the specification and claims are to be understood as being modified by the term “about”. Accordingly, unless indicated to the contrary, the numerical parameters set forth in the foregoing specification and attached claims are approximations that can vary depending upon the desired properties sought to be obtained by those skilled in the art utilizing the teachings disclosed herein.

Although specific embodiments have been illustrated and described herein, it will be appreciated by those of ordinary skill in the art that a variety of alternate and/or equivalent implementations can be substituted for the specific embodiments shown and described without departing from the scope of the present disclosure. This application is intended to cover any adaptations or variations of the specific embodiments discussed herein. Therefore, it is intended that this disclosure be limited only by the claims and the equivalents thereof.

1-17. (canceled)

18. A system for detecting extravasation into a tissue, the system comprising:
an article comprising:
a body comprising a first major surface, an opposing second major surface, a first side, and an opposing second side;
a first electrode disposed on the first major surface of the body, wherein the first electrode comprises at least one skin-penetrating microfeature;
a second electrode disposed on the first major surface of the body, wherein the second electrode comprises at least one skin-penetrating microfeature;
wherein the first electrode is electrically connected to the second electrode; and
a controller electrically connected to the first electrode and the second electrode, wherein the controller is configured to:
provide an input signal across the first electrode and the second electrode;
determine an output signal across the first electrode and the second electrode in response to the input signal, wherein the input signal ranges from about 400 kilohertz to about 1 megahertz;
determine at least one electrical parameter based on the output signal and the input signal; and
detect extravasation of a fluid into the tissue based on a change in the at least one electrical parameter.

19. The system of claim 18, wherein the controller is further configured to determine at least one of an extravasated volume of the fluid and a time period of extravasation based on the change in the at least one electrical parameter.

20. The system of claim 18, wherein the at least one electrical parameter comprises one or more of an impedance magnitude, an impedance phase angle, a capacitance, a resistance, and a reactance.

21. (canceled)

22. The system of claim 19, wherein, for the input signal having a frequency of about 1 megahertz, the first electrode and the second electrode provide a data set correlating a percentage change of the at least one electrical parameter to the extravasated volume of the fluid, and wherein a best linear fit to the data set has a slope of at least about 5% per milliliter of the fluid.

23. The system of claim 22, wherein the slope is from about 6% per milliliter of the fluid to about 9% per milliliter of the fluid.

24. The system of claim 22, wherein the best linear fit has a coefficient of determination of at least about 0.8.

25. The system of claim 18, wherein the controller is further configured to store a timestamp indicative of extravasation of the fluid in a memory.

26. The system of claim 18, wherein the controller is coupled to the article.

27. The system of claim 18, wherein the controller is spaced apart from the article.

28. The system of claim 18, wherein, upon detecting extravasation of the fluid, the controller is further configured to perform at least one of:
generating an alert; and
providing a stop signal to an infusion system to automatically stop infusion of the fluid.

29. A method for detecting extravasation, the method comprising:
providing an article comprising a body, a first electrode comprising at least one skin-penetrating microfeature and disposed on a first major surface of the body, and a second electrode comprising at least one skin-penetrating microfeature and disposed on the first major surface of the body, wherein the second electrode is spaced apart from the first electrode;
placing the first major surface of the body on an injection site, wherein the injection site is disposed between the first electrode and the second electrode;
providing an input signal across the first electrode and the second electrode, wherein the input signal ranges from about 400 kilohertz to about 1 megahertz determining an output signal across the first electrode and the second electrode in response to the input signal;
determining at least one electrical parameter based on the output signal and the input signal, wherein the at least one electrical parameter comprises one or more of an impedance magnitude, an impedance phase angle, a capacitance, a resistance, and a reactance; and
determining extravasation into a tissue based on a change of the at least one electrical parameter.

30. The method of claim 29, further comprising determining at least one of an extravasated volume of a fluid and a time period of extravasation based on the change in the at least one electrical parameter.

31. The method of claim 29, wherein the input signal has a frequency ranging from about 0 hertz to about 1 gigahertz.

32. The method of claim 30, further comprises determining, for the input signal having a frequency of about 1 megahertz, a data set correlating a percentage change of the at least one electrical parameter to the extravasated volume of the fluid, wherein a best linear fit to the data set has a slope of at least about 5% per milliliter of the fluid.

33. The method of claim 32, wherein the slope is from about 6% per milliliter of the fluid to about 9% per milliliter of the fluid.

34. The method of claim 32, wherein the best linear fit has a coefficient of determination of at least about 0.8.

35. (canceled)

36. (canceled)

37. The method of claim 29, further comprising determining the impedance magnitude for a frequency of the input signal ranging from about 400 kilohertz to about 1 megahertz.

38-39. (canceled)

40. The method of claim 29, further comprising determining the reactance for a frequency of the input signal of about 1 megahertz.

41. The method of claim 29, further comprising generating an alert upon detecting extravasation.

42. The method of claim 29, further comprising:
providing a securement device on a second major surface of the body; and
detachably coupling an intravenous catheter with the securement device.
